(19) United States
(12) Patent Application Publication
(10) Pub. No.: US 2026/0253723 A1
(43) Pub. Date: Aug. 27, 2026

(54) SYSTEM

(71) Applicant: SoftBank Group Corp., Tokyo (JP)

(72) Inventor: Haruka KARASAKI, Tokyo (JP)

(73) Assignee: SoftBank Group Corp., Tokyo (JP)

(21) Appl. No.: 19/534,792

(22) Filed: Feb. 10, 2026

(30) Foreign Application Priority Data
Feb. 21, 2025 (JP) 2025-027060

(51) Int. Cl. G16H 40/67; A61B 5/00; A61B 5/11; A61B 5/16; G06F 40/289; G06V 40/16; G10L 25/63

(52) U.S. Cl. CPC G16H 40/67; A61B 5/1112; A61B 5/165; A61B 5/7275; A61B 5/746; A61B 5/7475; G06F 40/289; G06V 40/174; G10L 25/63

(57) ABSTRACT

The system according to the embodiment comprises a collection unit, an analysis unit, a proposal unit, and a provision unit. The collection unit collects health data of a user. The analysis unit analyzes data collected by the collection unit. The proposal unit makes proposals based on analysis results obtained by the analysis unit. The provision unit provides the content proposed by the proposal unit to the user.

### CROSS-REFERENCE TO RELATED APPLICATION

The present application claims priority to and incorporates by reference the entire contents of Japanese Patent Application No. 2025-027060 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, health management for people with chronic diseases has not been sufficiently performed, and there is room for improvement.

### SUMMARY OF THE INVENTION

The system according to the embodiment comprises a collection unit, an analysis unit, a proposal unit, and a provision unit. The collection unit collects health data of a user. The analysis unit analyzes data collected by the collection unit. The proposal unit makes proposals based on analysis results obtained by the analysis unit. The provision unit provides the content proposed by the proposal unit to the user.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

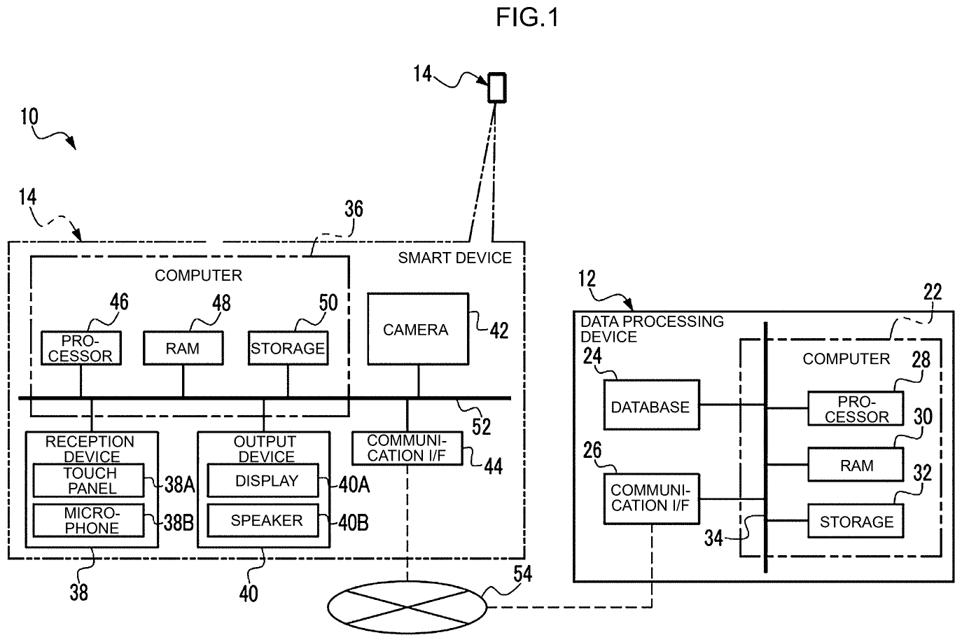

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

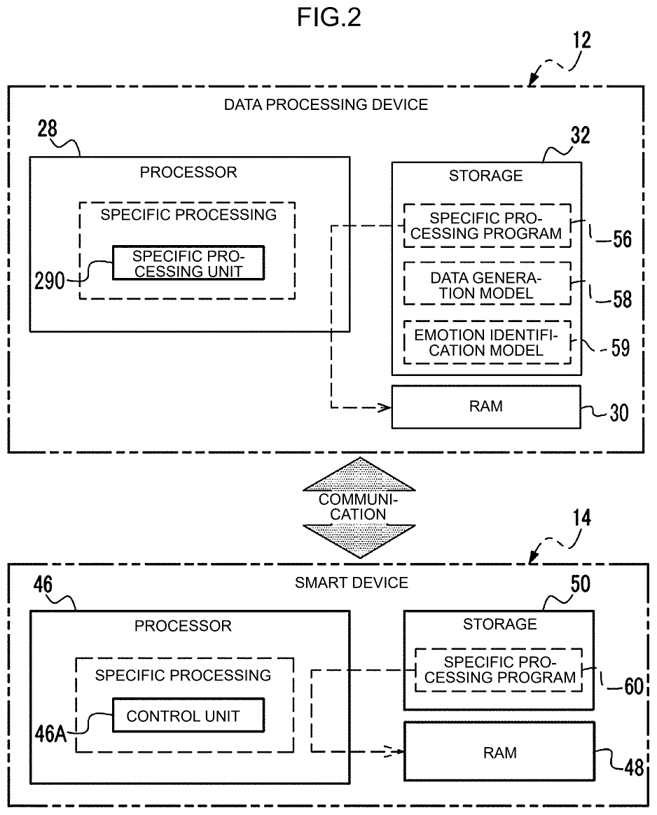

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management system according to the embodiment of the present invention is a system intended for people with chronic diseases such as chronic kidney disease, diabetes, and hypertension. This health management system collaborates with tools capable of measuring various numerical values such as blood glucose level and GFR (glomerular filtration rate) over time, and monitors the user's health condition in detail. The system accumulates data on what numerical fluctuations occurred when the user took certain actions, and proposes optimal choices for meals and actions according to the user's condition. The proposed content is output based on proprietary data obtained via the tools and AI training data. For example, the user uses a tool to measure numerical values such as blood glucose level and GFR. These values are automatically transmitted to the system and stored in a database. Next, the system analyzes the accumulated data and identifies the relationship between the user's behavior and numerical fluctuations. For example, if blood glucose level rises after consuming a specific meal, the system identifies the impact of that meal on blood glucose level. The system proposes optimal meals and actions based on the user's health condition. For example, if blood glucose level is high, the system can propose a low-carbohydrate meal and recommend exercise. These proposals are made based on proprietary data obtained via the tools and AI training data. The AI learns from past data and builds a model to make optimal proposals for the user's health condition. Furthermore, the system receives user feedback and improves the proposed content. By inputting the results of following the proposals into the system, the AI learns from these results and reflects them in future proposals. In this way, the system continuously supports the user's health management and provides optimal proposals. With this system, people with chronic diseases can understand their health condition in detail and take appropriate actions. For example, diabetic patients can grasp blood glucose fluctuations in real time and optimize their choices of meals and exercise. Kidney disease patients can monitor GFR fluctuations and take appropriate actions to maintain kidney function. Thus, the health management system can monitor the user's health condition in detail and provide optimal proposals. Specifically, this health management system acquires multidimensional health data such as blood glucose level, GFR, blood pressure, heart rate, and body temperature of the user in real time from wearable sensors and smartphone-linked devices. These data are stored in the database as time-series tensors (e.g., blood glucose level: 1,440 samples per day, float 32 per sample, 7 days as a [7,1440] two-dimensional tensor). The system also simultaneously collects behavioral data such as meal records (e.g., one-hot encoded vectors of meal content, numerical vectors for calorie intake and carbohydrate amount), exercise records (e.g., vectors for step count, calories burned, exercise intensity), and sleep records (e.g., sleep duration, sleep quality score). These diverse data undergo preprocessing such as missing value imputation, outlier removal, and normalization in the data preprocessing unit before being input to the analysis unit. In the analysis unit, for example, recurrent neural networks (RNN) for time-series analysis and multimodal Transformer architectures for integrating multiple data sources are used. Examples of inputs to the AI model include (1) one week of blood glucose time-series tensor, (2) recent meal content vector, (3) exercise intensity vector, (4) sleep score, and (5) user attributes (categorical data such as age, gender, disease history). The AI model outputs (a) prediction of blood glucose fluctuations for the next 24 hours (output example: 24-dimensional continuous value vector), (b) impact scores of meals, exercise, and sleep on blood glucose level and GFR (output example: contribution scores for each element, e.g., meal 0.6, exercise 0.3, sleep 0.1), and (c) specific options for recommended meals, exercise, and actions (output example: low-carb menu ID, recommended exercise type ID, recommended sleep duration). The AI model uses loss functions such as mean squared error (MSE) and cross-entropy loss, and optimizes weights by gradient descent. Training data consists of past user datasets (e.g., time-series data for 1,000 people over one year), and data augmentation such as noise addition and time-series shifting is applied. The output of the AI model is passed to the proposal unit, where threshold judgment (e.g., warning proposal if blood glucose prediction exceeds 180 mg/dL) and rule-based branching (e.g., prioritize kidney-protective meals when GFR decreases) are performed to generate user-specific proposed content. Furthermore, behavioral data on whether the user followed the proposal, changes in health indicators after the proposal, and subjective feedback from the user (e.g., proposal satisfaction score, free-text comments) are also collected and used for retraining and personalization of the AI model. As a technical effect, this system, unlike conventional simple health records or human advice, can analyze vast multidimensional data in real time and with high accuracy, and automatically generate optimized proposals for each user, thereby improving the accuracy of health management, enabling early detection of abnormalities, promoting behavioral change, and reducing medical costs. Specific application fields include home health management for diabetes, kidney disease, and hypertension patients, remote medical support, corporate health management platforms, and risk assessment systems for insurance companies.

The health management system according to the embodiment comprises a collection unit, an analysis unit, a proposal unit, and a provision unit. The collection unit collects health data of the user. The user's health data may include, for example, blood glucose level, GFR, blood pressure, heart rate, and body temperature, but is not limited to these examples. The collection unit can collect data using sensors, for example. The collection unit can also collect data manually entered by the user. For example, the collection unit can use a sensor for measuring blood glucose level to collect the user's blood glucose data. The collection unit can also use a sensor for measuring GFR to collect the user's GFR data. Furthermore, the collection unit can collect blood pressure data manually entered by the user. The analysis unit analyzes data collected by the collection unit. The analysis may be performed using statistical analysis or machine learning algorithms, for example, but is not limited to these examples. For example, the analysis unit can analyze trends in the user's health data using statistical analysis. The analysis unit can also analyze the user's health data using machine learning algorithms. Furthermore, the analysis unit can analyze correlations in the data. The proposal unit makes optimal proposals based on analysis results obtained by the analysis unit. Proposals may include, for example, proposals for meals or exercise, but are not limited to these examples. For example, the proposal unit can propose a low-carbohydrate meal based on the user's health condition. The proposal unit can also propose exercise based on the user's health condition. Furthermore, the proposal unit can propose stress management based on the user's health condition. The provision unit provides the content proposed by the proposal unit to the user. Provision may be performed by methods such as notifications, email, or in-app messages, but is not limited to these examples. For example, the provision unit can provide the proposed content to the user as a notification. The provision unit can also provide the proposed content to the user by email. Furthermore, the provision unit can provide the proposed content to the user as an in-app message. Thus, the health management system according to the embodiment can efficiently collect, analyze, propose, and provide the user's health data. Specifically, this health management system is equipped with multiple hardware interfaces for collecting the user's health data, such as wearable sensors, smartphone-linked devices, and home measurement devices. The collection unit acquires biosignals from these devices (e.g., blood glucose level recorded every minute as float32, GFR recorded daily as float32, blood pressure recorded twice daily as float32, heart rate recorded every second as int, body temperature recorded hourly as float32) as time-series tensors and stores them in the database. Furthermore, the collection unit simultaneously collects health data manually entered by the user via smartphone apps or web interfaces (e.g., meal content recorded as one-hot encoded vectors, exercise content recorded as category IDs, medication information recorded as binary flags). The collection unit automatically performs preprocessing such as missing value imputation (e.g., interpolation of recent values, median imputation), outlier removal (e.g., 3σ rule), and normalization (e.g., z-score normalization) to ensure data quality. The analysis unit inputs the multidimensional time-series data received from the collection unit into statistical analysis (e.g., moving average, analysis of variance), machine learning algorithms (e.g., random forest, support vector machine), and deep learning models (e.g., recurrent neural network, multimodal Transformer). Examples of inputs to the AI model include one week of blood glucose time-series tensor ([7,1440]), recent meal content vector ([20]), exercise intensity vector ([5]), sleep score (float), and user attributes (categorical data such as age, gender, disease history). The analysis unit outputs predictions of blood glucose fluctuations for the next 24 hours (output example: 24-dimensional continuous value vector), contribution scores of each element to health indicators (e.g., meal 0.6, exercise 0.3, sleep 0.1), and abnormal value detection flags (e.g., flag 1 for rapid increase in blood glucose level). The proposal unit receives the output results from the analysis unit and generates personalized proposals by rule-based branching (e.g., warning proposal if blood glucose prediction exceeds 180 mg/dL), AI-based optimization (e.g., behavioral proposals by reinforcement learning), and consideration of the user's past preferences, allergy information, family history, and genetic information. The proposal unit generates meal proposals (e.g., low-carb menu ID, recommended calorie intake), exercise proposals (e.g., 30 minutes walking, 10 minutes strength training), and stress management proposals (e.g., breathing techniques, use of meditation apps). The provision unit selects the optimal provision method (e.g., push notification, email, in-app message) for delivery, considering the user's device information (e.g., smartphone, tablet, smartwatch), usage history, and emotion estimation results (e.g., simple notification during stress, detailed notification during relaxation). Furthermore, user feedback (e.g., proposal satisfaction score, implementation status, free-text comments) and behavioral data (e.g., blood glucose fluctuations after proposal) are also collected and used for retraining the AI model and continuous improvement of the proposed content. As a technical effect, this system, unlike conventional simple health records or human advice, can analyze vast multidimensional data in real time and with high accuracy, and automatically generate optimized proposals for each user, thereby improving the accuracy of health management, enabling early detection of abnormalities, promoting behavioral change, and reducing medical costs. Specific application fields include home health management for diabetes, kidney disease, and hypertension patients, remote medical support, corporate health management platforms, and risk assessment systems for insurance companies.

The collection unit can collect health data of blood glucose level or GFR. For example, the collection unit can use a sensor for measuring blood glucose level to collect the user's blood glucose data. For example, the collection unit attaches a blood glucose sensor to the user's skin and continuously measures blood glucose level. The collection unit can also use a sensor for measuring GFR to collect the user's GFR data. For example, the collection unit measures GFR through urine tests and collects the data. Furthermore, the collection unit can collect blood glucose data manually entered by the user. For example, the collection unit collects data when the user measures blood glucose level and manually enters the result. By collecting health data such as blood glucose level and GFR, the user's health condition can be monitored in detail. Some or all of the above-described processing in the collection unit may be performed using AI or without using AI. For example, the collection unit can input data obtained from the blood glucose sensor into a generative AI and have the generative AI perform data analysis. Specifically, the collection unit is equipped with multiple hardware modules such as wearable continuous blood glucose measurement sensors, home urine test devices, and smartphone-linked manual input interfaces, and acquires biosignals from these sources at high frequency and in multiple dimensions. The collection unit records blood glucose data every minute as float 32, stores 1,440 samples per day, and stores one week of data as a [7,1440] two-dimensional tensor in the database. GFR data is recorded daily or weekly as float32 and managed as a time-series array for each user. Manually entered data is recorded as numerical vectors or category IDs via smartphone apps or web forms. The collection unit automatically performs preprocessing such as missing value imputation (e.g., interpolation of recent values, median imputation), outlier removal (e.g., 3σ rule), and normalization (e.g., z-score normalization) to ensure data quality. When using AI, the collection unit can input continuous time-series data obtained from the blood glucose sensor (e.g., [1440] vector of float32 type), GFR value obtained from urine tests (e.g., single float32 value), and manually entered blood glucose value (e.g., single float32 value) into a convolutional neural network (CNN) for abnormal value detection or a recurrent neural network (RNN) for time-series analysis. Examples of inputs to the AI model include (1) one day of blood glucose time-series tensor [1440], (2) recent GFR value float32, and (3) manually entered blood glucose value float32. The AI model outputs (a) abnormal value detection flag (output example: 0 or 1), (b) data quality score (output example: continuous value from 0.0 to 1.0), and (c) time-series data after missing value imputation (output example: [1440] vector of float32 type). For example, if outliers are included in the blood glucose time-series, the AI model automatically removes or corrects the relevant samples and stores only high-quality data in the database. Thus, the collection unit, unlike conventional simple data recording, realizes automatic quality management, abnormal detection, and preprocessing of high-dimensional data by AI, and can provide highly reliable health data to subsequent analysis and proposal units. As a technical effect, the collection unit can collect and preprocess vast time-series data and data from diverse input channels in real time and with high accuracy, enabling early detection of changes in health condition and optimized health management for each user. Specific application fields include home blood glucose monitoring for diabetic patients, GFR management for kidney disease patients, remote medical support, and corporate health management platforms.

The analysis unit can analyze the collected data and identify the relationship between the user's behavior and numerical fluctuations. For example, the analysis unit analyzes the collected data using statistical analysis. For example, the analysis unit analyzes the user's blood glucose data and identifies the impact of specific meals on blood glucose level. The analysis unit can also use machine learning algorithms to identify the relationship between the user's behavior and numerical fluctuations. For example, the analysis unit analyzes the user's exercise data and blood glucose data and identifies the impact of exercise on blood glucose level. Furthermore, the analysis unit can analyze correlations in the data. For example, the analysis unit analyzes the user's meal data and blood pressure data and identifies the impact of meals on blood pressure. By identifying the relationship between the user's behavior and numerical fluctuations, the factors causing changes in health condition can be understood. Some or all of the above-described processing in the analysis unit may be performed using AI or without using AI. For example, the analysis unit can input the collected data into a generative AI and have the generative AI perform data analysis. Specifically, the analysis unit integrates multidimensional time-series data received from the collection unit (e.g., blood glucose [7,1440] tensor, daily GFR array, blood pressure [14] vector measured twice daily, heart rate [86,400] vector, body temperature [24] vector) and behavioral data (e.g., meal content one-hot vector [20], exercise intensity vector [5], sleep score float, user attribute categorical data), and inputs them into the analysis pipeline after data preprocessing. The analysis unit first executes moving average, analysis of variance, autocorrelation analysis, etc., in the statistical analysis module to extract trends and periodicity of health indicators. Next, machine learning algorithms such as random forest and support vector machine are used to quantitatively evaluate the correlation between behavioral elements such as meals, exercise, and sleep and fluctuations in health indicators. Furthermore, deep learning models such as recurrent neural networks (RNN) and multimodal Transformers are used to perform high-dimensional feature extraction and causal inference by integrating multiple data sources. Examples of inputs to the AI model include (1) one week of blood glucose time-series tensor [7,1440], (2) recent meal content vector [20], (3) exercise intensity vector [5], (4) sleep score float, and (5) user attribute categorical data. The AI model outputs (a) prediction of blood glucose fluctuations for the next 24 hours (output example: 24-dimensional continuous value vector), (b) impact scores of meals, exercise, and sleep on blood glucose level and GFR (output example: meal 0.6, exercise 0.3, sleep 0.1), (c) abnormal value detection flag (output example: flag 1 for rapid increase in blood glucose level), and (d) correlation coefficient matrix between behavioral elements (output example: meal-blood glucose 0.7, exercise-blood glucose −0.4, etc.). For example, if the user's blood glucose level rises sharply immediately after consuming a high-carbohydrate meal, the analysis unit accurately extracts the correlation between the meal content vector and the blood glucose time-series and quantifies the contribution of the meal element. The AI model uses loss functions such as mean squared error (MSE) and cross-entropy loss, and optimizes weights by gradient descent. Training data consists of past user datasets (e.g., time-series data for 1,000 people over one year), and data augmentation such as noise addition and time-series shifting is applied. The analysis unit passes the output results of the AI model to the subsequent proposal unit for threshold judgment and rule-based branching (e.g., warning proposal if blood glucose prediction exceeds 180 mg/dL). As a technical effect, the analysis unit, unlike conventional simple statistical analysis or human trend recognition, can analyze vast multidimensional data in real time and with high accuracy, and automatically extract optimized factors causing changes in health condition for each user, thereby improving the accuracy of health management, enabling early detection of abnormalities, and promoting behavioral change. Specific application fields include home health management for diabetes, kidney disease, and hypertension patients, remote medical support, corporate health management platforms, and risk assessment systems for insurance companies.

The proposal unit can propose meals or actions based on the user's health condition. For example, the proposal unit proposes a low-carbohydrate meal based on the user's health condition. For example, the proposal unit proposes a low-carbohydrate meal when the user's blood glucose level is high. The proposal unit can also propose exercise based on the user's health condition. For example, the proposal unit proposes exercise when the user's blood pressure is high. Furthermore, the proposal unit can propose stress management based on the user's health condition. For example, the proposal unit proposes relaxation when the user's stress level is high. By making optimal proposals based on the user's health condition, health management can be supported. Some or all of the above-described processing in the proposal unit may be performed using AI or without using AI. For example, the proposal unit can input the user's health data into a generative AI and have the generative AI generate optimal proposals. Specifically, the proposal unit integrates health indicator prediction values received from the analysis unit (e.g., blood glucose prediction vector [24] for the next 24 hours, GFR prediction value float, blood pressure prediction vector [2]), contribution scores of each element (e.g., meal 0.6, exercise 0.3, sleep 0.1), abnormal value detection flags, user attributes (age, gender, disease history), preference and allergy information (e.g., allergen ID list, preferred ingredient ID list), family history and genetic information (e.g., disease risk score), and generates optimal proposals for meals, exercise, and stress management. Examples of inputs to the AI model include (1) blood glucose prediction vector [24], (2) GFR prediction value float, (3) meal contribution score float, (4) user preference vector [10], (5) allergy ID list, and (6) family history risk score float. The AI model outputs (a) recommended meal menu ID (output example: low-carb menu ID 123), (b) recommended exercise type ID (output example: walking ID 5), (c) recommended stress management method ID (output example: meditation app ID 7), (d) recommended calorie intake float, and (e) recommended exercise duration float. For example, if the blood glucose prediction is high and the user has a wheat allergy, the proposal unit prioritizes proposing a low-carb, wheat-free menu ID. The AI model uses reinforcement learning and multi-objective optimization algorithms to personalize the proposed content by reflecting the user's past behavioral history and feedback. The proposal unit passes the output results of the AI model to the subsequent provision unit and selects the optimal provision method according to the user's device information and emotional state. As a technical effect, the proposal unit, unlike conventional rule-based or human advice, can integrate vast multidimensional data and individual attributes and generate automatically optimized and personalized proposals in real time by AI, thereby improving the accuracy of health management, promoting behavioral change, and reducing medical costs. Specific application fields include home health management for diabetes, kidney disease, and hypertension patients, remote medical support, corporate health management platforms, and risk assessment systems for insurance companies.

The provision unit can provide the proposed content to the user. For example, the provision unit provides the proposed content to the user as a notification. For example, the provision unit sends a notification to the user's smartphone and provides the proposed content. The provision unit can also provide the proposed content to the user by email. For example, the provision unit sends the proposed content to the user's email address. Furthermore, the provision unit can provide the proposed content to the user as an in-app message. For example, the provision unit sends a message to the health management app used by the user and provides the proposed content. By providing the proposed content to the user, the user can take appropriate actions. Some or all of the above-described processing in the provision unit may be performed using AI or without using AI. For example, the provision unit can input the proposed content into a generative AI and have the generative AI execute the selection of the optimal provision method. Specifically, the provision unit integrates the proposed content received from the proposal unit, such as recommended meal menu ID, exercise type ID, stress management method ID, recommended calorie intake, and recommended exercise duration, with the user's device information (e.g., smartphone, tablet, smartwatch), usage history (e.g., past notification open rate, email viewing history), and emotion estimation results (e.g., simple notification during stress, detailed notification during relaxation), and selects the optimal provision method. Examples of inputs to the AI model include (1) proposed content ID list, (2) user device type ID, (3) emotional state label (e.g., stress, relaxation), and (4) past notification open rate float. The AI model outputs (a) provision method ID (output example: push notification ID 1, email ID 2, in-app message ID 3), (b) notification timing (output example: immediate, 8 a.m., 8 p.m.), and (c) level of detail of notification content (output example: summary only, with detailed explanation). For example, if the user is using a smartwatch and is in a stress state, the provision unit immediately sends a simple push notification. The AI model automatically selects the optimal provision channel, timing, and content by considering the user's past response history and emotional state. The provision unit generates actual notifications, emails, and in-app messages based on the output results of the AI model and delivers them to the user's device. As a technical effect, the provision unit, unlike conventional uniform information provision, can realize personalized information provision by considering the user's device characteristics, usage history, and emotional state, thereby improving the user's behavioral change rate, information transmission efficiency, and continuity of health management. Specific application fields include home health management apps, remote medical support systems, corporate health management platforms, and risk notification systems for insurance companies.

The proposal unit can receive user feedback and improve the proposed content. For example, the proposal unit collects user feedback through questionnaires. For example, the proposal unit sends a questionnaire to the user and collects feedback on the proposed content. The proposal unit can also collect feedback based on the user's behavioral data. For example, the proposal unit determines from behavioral data whether the user followed the proposal and collects the result as feedback. Furthermore, the proposal unit can collect feedback based on the user's emotional data. For example, the proposal unit analyzes the user's emotional data and collects emotional responses to the proposed content as feedback. By reflecting user feedback, the accuracy of the proposed content is improved. Emotion estimation is realized using, for example, an emotion engine or generative AI with emotion estimation functions. Generative AI may be a text generation AI (e.g., LLM) or a multimodal generative AI, but is not limited to these examples. Some or all of the above-described processing in the proposal unit may be performed using AI or without using AI. For example, the proposal unit can input user feedback data into a generative AI and have the generative AI execute improvement of the proposed content. Specifically, the proposal unit integrates feedback data collected from the user (e.g., proposal satisfaction score float, free-text comment, implementation status binary, post-proposal health indicator change vector, emotion estimation label) and realizes continuous improvement of the proposed content by the AI model. Examples of inputs to the AI model include (1) proposal satisfaction score float, (2) free-text comment, (3) implementation status binary, (4) post-proposal blood glucose fluctuation vector [24], and (5) emotion estimation label (e.g., positive, negative). The AI model outputs (a) improvement points for the proposed content in text (output example: diversification of meal proposals, adjustment of exercise proposal intensity), (b) parameter update values for the next proposal (output example: recommended calorie −50 kcal, exercise duration +10 minutes), and (c) weight updates for the user preference model (output example: increased weight for preferred ingredient ID). For example, if the user shows a negative emotion toward a low-carb menu proposal, the AI model adjusts the weights of the preference model and generates a proposal prioritizing preferred ingredients next time. The AI model uses natural language processing algorithms (e.g., BERT, Transformer) to analyze free-text comments and automatically extract improvement points for the proposed content. The proposal unit dynamically updates the parameters of the proposal generation algorithm based on the output results of the AI model and continuously optimizes personalized proposed content for each user. As a technical effect, the proposal unit, unlike conventional one-way advice provision, automatically analyzes and reflects subjective and objective user feedback using AI, thereby improving the accuracy of the proposed content, user satisfaction, and sustainability of behavioral change. Specific application fields include home health management apps, remote medical support systems, corporate health management platforms, and risk notification systems for insurance companies.

The collection unit can estimate the user's emotion and adjust the timing of health data collection based on the estimated emotion of the user. For example, if the user is feeling stressed, the collection unit collects blood glucose level and GFR data at a relaxed timing. For example, the collection unit photographs the user's facial expression with a camera and estimates emotion using an emotion estimation algorithm. If the user is relaxed, the collection unit can also collect health data at regular intervals. For example, the collection unit records the user's voice and estimates emotion using voice analysis technology. Furthermore, if the user is in a hurry, the collection unit can adjust to collect data in a short time. For example, the collection unit collects the user's biosignals (heart rate and skin conductance) with sensors and estimates emotion using an emotion estimation algorithm. By adjusting the collection timing according to the user's emotion, more accurate data can be collected. Emotion estimation is realized using, for example, an emotion engine or generative AI with emotion estimation functions. Generative AI may be a text generation AI (e.g., LLM) or a multimodal generative AI, but is not limited to these examples. Some or all of the above-described processing in the collection unit may be performed using AI or without using AI. For example, the collection unit can input the user's emotional data into a generative AI and have the generative AI execute adjustment of the collection timing. Specifically, the collection unit simultaneously acquires multiple sensor data (e.g., camera image [224,224,3] tensor, audio waveform [16,000] array, heart rate time-series [60] vector, skin conductance [60] vector) to estimate the user's emotional state. The collection unit normalizes, removes noise, and extracts features (e.g., facial expression feature vector [128], audio spectral feature [64], heart rate variability index float, skin conductance change float) in a multimodal preprocessing unit and inputs them into an emotion estimation AI model. The emotion estimation AI model adopts a hybrid configuration of multimodal Transformer and convolutional neural network (CNN) plus recurrent neural network (RNN), and receives as input (1) facial expression feature vector [128], (2) audio feature vector [64], (3) heart rate variability float, and (4) skin conductance float. The AI model outputs (a) emotion label (output example: stress, relaxation, tension, excitement), (b) emotion intensity score (output example: continuous value from 0.0 to 1.0), and (c) recommended collection timing (output example: immediate, 5 minutes later, 30 minutes later). For example, if the user is estimated to be in a stress state (emotion label: stress, intensity 0.8), the collection unit delays the collection of blood glucose level and GFR data by 30 minutes. Conversely, if the user is in a relaxed state (emotion label: relaxation, intensity 0.2), data is collected at regular intervals. The AI model uses loss functions such as cross-entropy loss and mean squared error to maximize emotion estimation accuracy and is trained on GPU clusters. Training data consists of multimodal biosignal datasets with emotion labels (e.g., 10,000 samples), and data augmentation such as rotation of facial images, pitch shift of audio, and noise addition to heart rate is applied. The output of the AI model is passed to the collection timing control module, where threshold judgment (e.g., stress intensity 0.7 or higher delays collection) and rule-based branching (e.g., immediate collection of minimum data when in a hurry) are performed and reflected in the actual data collection scheduler. As a technical effect, the collection unit, unlike conventional uniform data collection, can estimate the user's emotional state with high accuracy and in real time, and dynamically optimize the collection timing, thereby simultaneously reducing user burden and improving data quality. This reduces noise and measurement errors in biosignals under stress and greatly improves the analysis accuracy of health management AI. Specific application fields include home health monitoring, remote medical support, corporate stress management systems, and condition management for athletes.

The collection unit can analyze the user's past health data and select a collection method. For example, the collection unit analyzes the user's past blood glucose data and collects data at the most stable timing. For example, the collection unit analyzes blood glucose data from the past year and identifies time periods when blood glucose level is stable. The collection unit can also analyze the user's GFR data and collect data during periods of low fluctuation. For example, the collection unit analyzes past GFR data and identifies time periods when GFR is stable. Furthermore, the collection unit can determine the optimal collection frequency based on the user's past health data. For example, the collection unit analyzes past health data and identifies the optimal collection frequency. By analyzing past data, the optimal collection method can be selected. Some or all of the above-described processing in the collection unit may be performed using AI or without using AI. For example, the collection unit can input past health data into a generative AI and have the generative AI execute selection of the collection method. Specifically, the collection unit acquires past one year of health data for each user (e.g., blood glucose [365,1440] tensor, GFR [365] vector, blood pressure vector) from the database and inputs them into a time-series analysis module. The collection unit first uses statistical methods such as moving average, variance, and autocorrelation analysis to extract stable time periods for blood glucose level and GFR (e.g., daytime 9-11 a.m., nighttime 10-12 p.m.). Next, machine learning algorithms (e.g., k-means clustering, self-organizing maps) are used to classify data fluctuation patterns and identify time periods belonging to stable clusters. Furthermore, deep learning models (e.g., time-series LSTM networks) are used to predict future data fluctuations and estimate optimal collection timing. Examples of inputs to the AI model include (1) one year of blood glucose time-series tensor [365,1440], (2) daily GFR vector [365], and (3) morning and evening blood pressure vector [365,2]. The AI model outputs (a) stable time period labels (output example: 9-11 a.m., 10-12 p.m.), (b) optimal collection frequency (output example: twice daily, three times weekly), and (c) fluctuation prediction score (output example: continuous value from 0.1 to 1.0). For example, if blood glucose level is stable after breakfast and dinner, the collection unit concentrates data collection during those time periods. The AI model uses loss functions such as mean squared error and clustering loss to maximize pattern recognition accuracy. Training data consists of long-term health datasets from many users, and data augmentation such as time-series shifting and noise addition is applied. The output of the AI model is passed to the collection scheduler, where rule-based branching (e.g., increase frequency when fluctuation is large, decrease frequency when stable) and threshold judgment are performed and reflected in the actual data collection plan. As a technical effect, the collection unit, unlike conventional fixed-interval collection, dynamically optimizes collection timing, frequency, and method based on analysis of past data for each user, thereby reducing unnecessary data collection and accurately capturing important fluctuations. This enables efficient database operation, reduces measurement burden, and improves the analysis accuracy of health management AI. Specific application fields include home monitoring for chronic disease patients, remote medical support, corporate health management systems, and risk assessment for insurance companies.

The collection unit can perform filtering based on the user's current living situation or areas of interest at the time of health data collection. For example, if the user is exercising, the collection unit collects blood glucose level at the optimal timing after exercise. For example, the collection unit analyzes the user's exercise data and collects blood glucose level at the optimal timing after exercise. The collection unit can also collect GFR after meals if the user is eating. For example, the collection unit analyzes the user's meal data and collects GFR at the optimal timing after meals. Furthermore, if the user is relaxed, the collection unit can preferentially collect data during relaxation. For example, the collection unit analyzes the user's relaxation data and collects data at the optimal timing during relaxation. By filtering data based on the user's living situation or areas of interest, highly relevant data can be collected. Some or all of the above-described processing in the collection unit may be performed using AI or without using AI. For example, the collection unit can input the user's living situation data into a generative AI and have the generative AI execute data filtering. Specifically, the collection unit acquires the user's behavioral data (e.g., exercise record [10] vector, meal record [20] vector, relaxation state label, activity log time-series [1440]) in real time and inputs them into a behavior recognition AI model. The collection unit first uses a behavior recognition module (e.g., convolutional neural network plus LSTM) to classify states such as exercise, meal, and relaxation with high accuracy. Examples of inputs to the AI model include (1) activity log for the past hour [60], (2) meal content vector [20], (3) exercise intensity vector [5], and (4) relaxation state label. The AI model outputs (a) current behavioral state label (output example: exercising, eating, relaxing), (b) optimal collection timing (output example: 5 minutes after exercise, 30 minutes after meals, during relaxation), and (c) collection priority score (output example: continuous value from 0.0 to 1.0). For example, if the user is determined to be exercising, the collection unit schedules blood glucose collection 5 minutes after exercise. If eating, GFR is collected 30 minutes after meals. If relaxing, heart rate and body temperature are preferentially collected at that timing. The AI model uses loss functions such as cross-entropy loss and mean squared error to maximize behavior recognition accuracy and collection optimization accuracy. Training data consists of behavioral and health datasets from users with diverse living patterns, and data augmentation such as shuffling of behavior labels and noise addition is applied. The output of the AI model is passed to the collection scheduler, where rule-based branching (e.g., prioritize blood glucose after exercise, prioritize GFR after meals) and threshold judgment are performed and reflected in the actual data collection plan. As a technical effect, the collection unit, unlike conventional uniform data collection, can recognize the user's real-time living situation and areas of interest with high accuracy and preferentially collect only highly relevant data, thereby greatly improving the usefulness of data and analysis accuracy. Specific application fields include behavior-linked monitoring for lifestyle disease patients, personalized health management, performance management for athletes, and corporate health management support.

The collection unit can estimate the user's emotion and determine the priority of health data to be collected based on the estimated emotion of the user. For example, if the user is feeling stressed, the collection unit preferentially collects data related to stress. For example, the collection unit photographs the user's facial expression with a camera and estimates emotion using an emotion estimation algorithm. If the user is relaxed, the collection unit can also collect overall health data in a balanced manner. For example, the collection unit records the user's voice and estimates emotion using voice analysis technology. Furthermore, if the user is in a hurry, the collection unit can preferentially collect only important data. For example, the collection unit collects the user's biosignals (heart rate and skin conductance) with sensors and estimates emotion using an emotion estimation algorithm. By determining the priority of data according to the user's emotion, important data can be preferentially collected. Emotion estimation is realized using, for example, an emotion engine or generative AI with emotion estimation functions. Generative AI may be a text generation AI (e.g., LLM) or a multimodal generative AI, but is not limited to these examples. Some or all of the above-described processing in the collection unit may be performed using AI or without using AI. For example, the collection unit can input the user's emotional data into a generative AI and have the generative AI execute determination of data priority. Specifically, the collection unit simultaneously acquires multiple biosignals (e.g., facial expression image [224,224,3], audio waveform [16,000], heart rate [60], skin conductance [60]) and inputs them into an emotion estimation AI model. The collection unit uses the emotion estimation AI model (e.g., multimodal Transformer, CNN plus RNN) to output emotion labels (e.g., stress, relaxation, tension, excitement) and emotion intensity scores (0.0-1.0). Examples of inputs to the AI model include (1) facial expression feature vector [128], (2) audio feature vector [64], (3) heart rate variability float, and (4) skin conductance float. The AI model outputs (a) emotion label, (b) emotion intensity score, and (c) recommended collection data priority list (output example: prioritize heart rate and skin conductance during stress, collect blood glucose level, GFR, blood pressure, and body temperature in a balanced manner during relaxation, collect only blood glucose level and GFR when in a hurry). For example, if the user is estimated to be in a stress state (emotion label: stress, intensity 0.8), the collection unit preferentially collects stress-related data such as heart rate and skin conductance. In a relaxed state, overall health data is collected in a balanced manner. When in a hurry, only important data (e.g., blood glucose level, GFR) is preferentially collected. The AI model uses loss functions such as cross-entropy loss and mean squared error to maximize emotion estimation accuracy and priority determination accuracy. Training data consists of multimodal biosignal datasets with emotion labels, and data augmentation such as rotation of facial images, noise addition to audio, and variability addition to heart rate is applied. The output of the AI model is passed to the collection priority control module, where rule-based branching (e.g., prioritize stress-related data when stress intensity is 0.7 or higher) and threshold judgment are performed and reflected in the actual data collection plan. As a technical effect, the collection unit, unlike conventional uniform data collection, can estimate the user's emotional state with high accuracy and dynamically optimize the priority of data to be collected, thereby preventing the oversight of important health indicators and minimizing user burden. Specific application fields include stress management systems, home health monitoring, remote medical support, and corporate health management platforms.

The collection unit can preferentially collect highly relevant data based on the user's geographic location information at the time of health data collection. For example, if the user is at a high altitude, the collection unit preferentially collects data related to oxygen concentration. For example, the collection unit acquires the user's geographic location information and collects oxygen concentration data when the user is at a high altitude. The collection unit can also preferentially collect data related to environmental pollution when the user is in an urban area. For example, the collection unit acquires the user's geographic location information and collects environmental pollution data when the user is in an urban area. Furthermore, if the user is at a sports facility, the collection unit can preferentially collect data related to exercise. For example, the collection unit acquires the user's geographic location information and collects exercise data when the user is at a sports facility. By considering geographic location information, highly relevant data can be collected. Some or all of the above-described processing in the collection unit may be performed using AI or without using AI. For example, the collection unit can input the user's geographic location information into a generative AI and have the generative AI execute data collection. Specifically, the collection unit acquires the user's location information (e.g., GPS coordinates [2], altitude float, facility ID) in real time and inputs them into a geographic information analysis AI model. The collection unit uses the geographic information analysis AI model (e.g., location embedding plus fully connected network) to classify the characteristics of the user's current location (e.g., high altitude, urban area, sports facility, residential area) and estimate the types of health data with high relevance. Examples of inputs to the AI model include (1) GPS coordinates [2], (2) altitude float, (3) facility ID, and (4) surrounding environment data vector [10]. The AI model outputs (a) current location category (output example: high altitude, urban area, sports facility), (b) recommended collection data type list (output example: oxygen concentration and heart rate at high altitude, environmental pollution and respiratory function in urban areas, exercise intensity and calories burned at sports facilities), and (c) collection priority score (output example: continuous value from 0.0 to 1.0). For example, if the user is at a high altitude of 2,000 m, the collection unit preferentially collects data such as oxygen concentration and heart rate. In urban areas, environmental pollution indicators and respiratory function data are prioritized. At sports facilities, data such as exercise intensity and calories burned are preferentially collected. The AI model uses loss functions such as cross-entropy loss and mean squared error to maximize geographic information classification accuracy and collection optimization accuracy. Training data consists of health datasets from diverse geographic environments, and data augmentation such as noise addition to location information and variability addition to environmental data is applied. The output of the AI model is passed to the collection scheduler, where rule-based branching (e.g., prioritize oxygen concentration at high altitude, prioritize environmental pollution in urban areas) and threshold judgment are performed and reflected in the actual data collection plan. As a technical effect, the collection unit, unlike conventional uniform data collection, can analyze the user's geographic location information with high accuracy and preferentially collect health data with high relevance according to the environment, thereby enabling early detection of health risks due to environmental factors and personalized health management. Specific application fields include health monitoring for high-altitude climbers, environmental risk management for urban residents, performance management for sports facility users, and remote medical support.

The collection unit can analyze the user's social media activity and collect relevant data at the time of health data collection. For example, if the user is feeling stressed on social media, the collection unit collects data related to stress. For example, the collection unit analyzes the user's social media posts and collects data related to stress. The collection unit can also collect data related to health if the user shares health information on social media. For example, the collection unit analyzes the user's social media posts and collects health-related information. Furthermore, if the user shares information about exercise on social media, the collection unit can collect data related to exercise. For example, the collection unit analyzes the user's social media posts and collects data related to exercise. By analyzing social media activity, relevant data can be collected. Some or all of the above-described processing in the collection unit may be performed using AI or without using AI. For example, the collection unit can input the user's social media data into a generative AI and have the generative AI execute data collection. Specifically, the collection unit acquires the user's social media post data (e.g., text posts, images, videos, posting time, number of likes, number of comments) via API and inputs them into a natural language processing AI model and an image analysis AI model. The collection unit uses a natural language processing AI model (e.g., Transformer-based text classifier) to classify post text into categories such as stress, health, exercise, and irrelevant with high accuracy. Examples of inputs to the AI model include (1) post text (up to 512 tokens), (2) post image feature vector [256], (3) posting time, and (4) engagement indicators (number of likes, number of comments). The AI model outputs (a) post category label (output example: stress, health, exercise, irrelevant), (b) emotion score (output example: continuous value from −1.0 to 1.0), and (c) recommended collection data type list (output example: collect heart rate and skin conductance for stress posts, collect blood glucose level and GFR for health posts, collect exercise intensity and calories burned for exercise posts). For example, if the user posts “I've been busy at work and feeling tired lately,” the AI model outputs the stress category and a high stress emotion score, and the collection unit prioritizes collecting stress-related data. For health-related posts, health indicators such as blood glucose level and GFR are collected. For exercise-related posts, data such as exercise intensity and calories burned are collected. The AI model uses loss functions such as cross-entropy loss and emotion regression loss to maximize post classification accuracy and emotion estimation accuracy. Training data consists of labeled social media post datasets, and data augmentation such as paraphrase generation for text and rotation or noise addition for images is applied. The output of the AI model is passed to the collection scheduler, where rule-based branching (e.g., prioritize stress-related data for stress posts) and threshold judgment are performed and reflected in the actual data collection plan. As a technical effect, the collection unit, unlike conventional user self-reporting or periodic collection, can analyze social media activity with high accuracy, detect psychological and behavioral changes of the user in real time, and automatically collect highly relevant health data, thereby enabling early detection of behavioral changes and personalized health management. Specific application fields include stress management, prevention of lifestyle diseases, corporate health management support, and remote medical monitoring.

The analysis unit can estimate the user's emotion and adjust the method of presenting analysis based on the estimated emotion of the user. For example, if the user is nervous, the analysis unit provides simple and highly visible analysis results. For example, the analysis unit photographs the user's facial expression with a camera and estimates emotion using an emotion estimation algorithm. If the user is relaxed, the analysis unit can also provide detailed analysis results. For example, the analysis unit records the user's voice and estimates emotion using voice analysis technology. Furthermore, if the user is in a hurry, the analysis unit can provide analysis results that focus on key points. For example, the analysis unit collects the user's biosignals (heart rate and skin conductance) with sensors and estimates emotion using an emotion estimation algorithm. By adjusting the method of presenting analysis according to the user's emotion, more appropriate analysis results can be provided. Emotion estimation is realized using, for example, an emotion engine or generative AI with emotion estimation functions. Generative AI may be a text generation AI (e.g., LLM) or a multimodal generative AI, but is not limited to these examples. Some or all of the above-described processing in the analysis unit may be performed using AI or without using AI. For example, the analysis unit can input the user's emotional data into a generative AI and have the generative AI execute adjustment of the method of presenting analysis. Specifically, the analysis unit simultaneously acquires multiple sensor data (e.g., facial expression image [224,224,3] tensor, audio waveform [16,000] array, heart rate time-series [60] vector, skin conductance [60] vector) to estimate the user's emotional state, normalizes, removes noise, and extracts features (e.g., facial expression feature vector [128], audio spectral feature [64], heart rate variability index float, skin conductance change float) in a multimodal preprocessing unit, and inputs them into an emotion estimation AI model. The analysis unit adopts a hybrid configuration of multimodal Transformer and convolutional neural network (CNN) plus recurrent neural network (RNN) as the emotion estimation AI model, and receives as input (1) facial expression feature vector [128], (2) audio feature vector [64], (3) heart rate variability float, and (4) skin conductance float. The AI model outputs (a) emotion label (output example: nervous, relaxed, in a hurry), and (b) emotion intensity score (output example: continuous value from 0.0 to 1.0). For example, if the user is estimated to be in a nervous state (emotion label: nervous, intensity 0.7), the analysis unit automatically generates a simple UI that emphasizes only the key points of graphs and numerical values as the method of presenting analysis results. In a relaxed state (emotion label: relaxed, intensity 0.2), detailed graphs, explanations of time-series fluctuations, and factor analysis comments are included in a rich analysis result. When in a hurry, only the key points are presented in a bulleted list. The AI model uses loss functions such as cross-entropy loss and mean squared error to maximize emotion estimation accuracy and is trained on GPU clusters. Training data consists of multimodal biosignal datasets with emotion labels (e.g., 10,000 samples), and data augmentation such as rotation of facial images, pitch shift of audio, and noise addition to heart rate is applied. The output of the AI model is passed to the analysis result generation module, where threshold judgment (e.g., simple display for nervous intensity 0.6 or higher) and rule-based branching (e.g., detailed display during relaxation) are performed and reflected in the actual analysis result UI or report generation. As a technical effect, the analysis unit, unlike conventional uniform analysis result display, can estimate the user's emotional state with high accuracy and in real time, and dynamically optimize the method of presenting analysis results, thereby greatly improving the user's understanding, satisfaction, and behavioral change rate. This prevents confusion due to information overload under stress and dissatisfaction due to lack of information during relaxation, and dramatically improves the user experience of health management AI. Specific application fields include home health monitoring apps, remote medical support systems, corporate health management platforms, and condition management for athletes.

The analysis unit can adjust the level of detail of analysis based on the importance of the health data during analysis. For example, the analysis unit performs detailed analysis for important health data. For example, the analysis unit analyzes the user's blood glucose data in detail and identifies factors causing blood glucose fluctuations. The analysis unit can also perform simplified analysis for general health data. For example, the analysis unit simplifies and analyzes the user's body temperature data. Furthermore, the analysis unit can adjust the level of detail of analysis according to the user's health condition. For example, the analysis unit performs detailed analysis when the user's health risk is high. By adjusting the level of detail of analysis based on the importance of health data, detailed analysis can be performed for important data. Some or all of the above-described processing in the analysis unit may be performed using AI or without using AI. For example, the analysis unit can input health data into a generative AI and have the generative AI execute adjustment of the level of detail of analysis. Specifically, the analysis unit normalizes, imputes missing values, and removes outliers from multidimensional health data received from the collection unit (e.g., blood glucose [7,1440] tensor, GFR [7] vector, blood pressure [14] vector, heart rate [86,400] vector, body temperature [24] vector) in the data preprocessing unit and inputs them into an importance judgment module. The analysis unit uses rule-based methods (e.g., high importance if blood glucose fluctuation exceeds threshold) or machine learning models (e.g., abnormal score estimation by random forest) as the importance judgment module to calculate importance scores for each health indicator (output example: blood glucose 0.9, GFR 0.8, body temperature 0.3). Examples of inputs to the AI model include (1) time-series data for each health indicator, (2) user attributes (age, disease history), and (3) recent abnormal value detection flags. The AI model outputs (a) importance score (0.0-1.0), and (b) recommended level of detail of analysis (output example: detailed, standard, simplified). For example, if the importance score for blood glucose is high (0.9), the analysis unit uses a recurrent neural network (RNN) or autoregressive model to perform detailed factor analysis of fluctuations (e.g., estimation of contribution of meals, exercise, and sleep) and generates a detailed report including graphs and factor explanations. If the importance score for body temperature is low (0.3), only simple moving averages or distribution summaries are presented. If health risk is high (e.g., sustained high blood glucose over the past week), detailed analysis is forced for all indicators. The AI model uses loss functions such as importance classification loss and analysis accuracy loss to minimize oversight of important data. Training data consists of health datasets with importance labels by medical experts, and data augmentation such as addition of abnormal values and time-series shifting is applied. The output of the AI model is passed to the analysis pipeline control module, where rule-based branching (e.g., detailed analysis for importance 0.7 or higher) and threshold judgment are performed and reflected in the actual analysis flow. As a technical effect, the analysis unit, unlike conventional uniform analysis processing, can accurately determine the importance of health data and concentrate computational resources on important data, thereby simultaneously improving analysis accuracy, computational efficiency, and preventing information overload for users. This greatly improves early detection of abnormalities in medical settings and home health management, and the quality of decision support for users. Specific application fields include home monitoring for chronic disease patients, remote medical support, corporate health management platforms, and risk assessment systems for insurance companies.

The analysis unit can apply different analysis algorithms according to the category of health data during analysis. For example, the analysis unit applies a dedicated analysis algorithm for blood glucose data. For example, the analysis unit analyzes blood glucose data using regression analysis. The analysis unit can also apply a dedicated analysis algorithm for GFR data. For example, the analysis unit analyzes GFR data using clustering. Furthermore, the analysis unit can apply a dedicated analysis algorithm for blood pressure data. For example, the analysis unit analyzes blood pressure data using time-series analysis. By applying different analysis algorithms according to the category of health data, more accurate analysis is possible. Some or all of the above-described processing in the analysis unit may be performed using AI or without using AI. For example, the analysis unit can input health data into a generative AI and have the generative AI execute application of analysis algorithms. Specifically, the analysis unit inputs multidimensional health data received from the collection unit (e.g., blood glucose [7,1440] tensor, GFR [7] vector, blood pressure [14] vector, heart rate [86,400] vector, body temperature [24] vector) into a data category judgment module and automatically selects the optimal analysis algorithm for each data type. For blood glucose data, time-series regression models (e.g., LSTM networks or autoregressive models) and fluctuation factor analysis algorithms (e.g., estimation of contribution of meals, exercise, and sleep) are applied. For GFR data, clustering (e.g., k-means, self-organizing maps) and abnormal value detection algorithms (e.g., Isolation Forest) are applied. For blood pressure data, time-series analysis (e.g., autoregressive moving average model ARMA, recurrent neural network) and periodicity detection algorithms are applied. Examples of inputs to the AI model include (1) blood glucose time-series tensor [7,1440], (2) GFR vector [7], (3) blood pressure vector [14], and (4) user attribute categorical data. The AI model outputs (a) category judgment label (output example: blood glucose, GFR, blood pressure), (b) recommended analysis algorithm ID (output example: LSTM, k-means, ARMA), and (c) analysis result (e.g., blood glucose fluctuation prediction vector, GFR cluster label, blood pressure periodicity score). For example, LSTM is applied to blood glucose data for 24-hour prediction, clustering is applied to GFR data for classification into stable and unstable groups, and periodicity analysis is applied to blood pressure data for abnormal value detection. The AI model uses loss functions such as category classification loss and analysis accuracy loss to maximize optimal algorithm selection accuracy. Training data consists of health datasets with analysis algorithm labels by medical experts, and data augmentation such as shuffling of category labels and noise addition is applied. The output of the AI model is passed to the analysis pipeline control module, where rule-based branching (e.g., LSTM for blood glucose, k-means for GFR) and threshold judgment are performed and reflected in the actual analysis flow. As a technical effect, the analysis unit, unlike conventional uniform application of analysis algorithms, can automatically select and apply the optimal algorithm for each category of health data, thereby simultaneously improving analysis accuracy, computational efficiency, and abnormal value detection rate. This enables risk assessment for each disease in medical settings and home health management, and personalized analysis for each user. Specific application fields include home monitoring for chronic disease patients, remote medical support, corporate health management platforms, and risk assessment systems for insurance companies.

The analysis unit can estimate the user's emotion and adjust the length of analysis based on the estimated emotion of the user. For example, if the user is in a hurry, the analysis unit provides a short analysis result that focuses on key points. For example, the analysis unit photographs the user's facial expression with a camera and estimates emotion using an emotion estimation algorithm. If the user is relaxed, the analysis unit can also provide detailed analysis results. For example, the analysis unit records the user's voice and estimates emotion using voice analysis technology. Furthermore, if the user is excited, the analysis unit can provide visually stimulating analysis results. For example, the analysis unit collects the user's biosignals (heart rate and skin conductance) with sensors and estimates emotion using an emotion estimation algorithm. By adjusting the length of analysis according to the user's emotion, the analysis unit can provide optimal analysis results for the user. Emotion estimation is realized using, for example, an emotion engine or generative AI with emotion estimation functions. Generative AI may be a text generation AI (e.g., LLM) or a multimodal generative AI, but is not limited to these examples. Some or all of the above-described processing in the analysis unit may be performed using AI or without using AI. For example, the analysis unit can input the user's emotional data into a generative AI and have the generative AI execute adjustment of the length of analysis. Specifically, the analysis unit simultaneously acquires multiple sensor data (e.g., facial expression image [224,224,3] tensor, audio waveform [16,000] array, heart rate time-series [60] vector, skin conductance vector) to estimate the user's emotional state, normalizes, removes noise, and extracts features (e.g., facial expression feature vector [128], audio spectral feature [64], heart rate variability index float, skin conductance change float) in a multimodal preprocessing unit, and inputs them into an emotion estimation AI model. The emotion estimation AI model adopts a hybrid configuration of multimodal Transformer and CNN plus RNN, and receives as input (1) facial expression feature vector [128], (2) audio feature vector [64], (3) heart rate variability float, and (4) skin conductance float. The AI model outputs (a) emotion label (output example: in a hurry, relaxed, excited), and (b) emotion intensity score (output example: continuous value from 0.0 to 1.0). For example, if the user is estimated to be in a hurry (emotion label: in a hurry, intensity 0.8), the analysis unit automatically shortens the length of the analysis result and presents only the key points in a bulleted list or graph. In a relaxed state (emotion label: relaxed, intensity 0.2), detailed graphs, explanations of time-series fluctuations, and factor analysis comments are included in a long report. In an excited state, visually stimulating analysis results using colors and animations are generated. The AI model uses loss functions such as cross-entropy loss and mean squared error to maximize emotion estimation accuracy and optimization accuracy of analysis length, and is trained on GPU clusters. Training data consists of multimodal biosignal datasets with emotion labels (e.g., 10,000 samples), and data augmentation such as rotation of facial images, pitch shift of audio, and noise addition to heart rate is applied. The output of the AI model is passed to the analysis result generation module, where threshold judgment (e.g., shortened display for hurry intensity 0.7 or higher) and rule-based branching (e.g., detailed display during relaxation) are performed and reflected in the actual analysis result UI or report generation. As a technical effect, the analysis unit, unlike conventional uniform analysis result display, can estimate the user's emotional state with high accuracy and in real time, and dynamically optimize the length of analysis results, thereby greatly improving the user's understanding, satisfaction, and behavioral change rate. This prevents confusion due to information overload and dissatisfaction due to lack of information, and dramatically improves the user experience of health management AI. Specific application fields include home health monitoring apps, remote medical support systems, corporate health management platforms, and condition management for athletes.

The analysis unit can determine the priority of analysis based on the timing of health data collection during analysis. For example, the analysis unit preferentially analyzes recently collected data. For example, the analysis unit preferentially analyzes the latest blood glucose data. The analysis unit can also analyze current data with reference to past data. For example, the analysis unit analyzes current GFR data with reference to GFR data from the past year. Furthermore, the analysis unit can adjust the priority of analysis according to the user's health condition. For example, the analysis unit preferentially analyzes the latest data when the user's health risk is high. By determining the priority of analysis based on the timing of health data collection, the latest data can be preferentially analyzed. Some or all of the above-described processing in the analysis unit may be performed using AI or without using AI. For example, the analysis unit can input health data into a generative AI and have the generative AI execute determination of analysis priority. Specifically, the analysis unit acquires multidimensional health data received from the collection unit (e.g., blood glucose [365,1440] tensor, GFR [365] vector, blood pressure [365,2] vector) from the database and inputs them, together with data collection time information, into a priority judgment module. The analysis unit uses rule-based methods (e.g., latest data has high priority) or machine learning models (e.g., abnormal score estimation by random forest) as the priority judgment module to calculate analysis priority scores for each data (output example: latest blood glucose 0.9, GFR from one week ago 0.5). Examples of inputs to the AI model include (1) time-series data for each health indicator, (2) data collection time, (3) user attributes (age, disease history), and (4) recent abnormal value detection flags. The AI model outputs (a) analysis priority score (0.0-1.0), and (b) recommended analysis order list (output example: latest blood glucose→latest GFR→past blood pressure). For example, if health risk is high (e.g., sustained high blood glucose over the past week), the priority of the latest data is further increased for analysis. The AI model uses loss functions such as priority classification loss and analysis accuracy loss to minimize oversight of important data. Training data consists of health datasets with priority labels by medical experts, and data augmentation such as time-series shifting and addition of abnormal values is applied. The output of the AI model is passed to the analysis pipeline control module, where rule-based branching (e.g., latest data is always top priority) and threshold judgment are performed and reflected in the actual analysis flow. As a technical effect, the analysis unit, unlike conventional uniform analysis order, can dynamically optimize analysis priority according to the timing of health data collection and user condition, thereby simultaneously realizing early detection of abnormalities, rapid response, and efficient use of computational resources. This enables real-time risk management and personalized analysis for each user in medical settings and home health management. Specific application fields include home monitoring for chronic disease patients, remote medical support, corporate health management platforms, and risk assessment systems for insurance companies.

The analysis unit can adjust the order of analysis based on the relevance of health data during analysis. For example, the analysis unit preferentially analyzes highly relevant data. For example, the analysis unit analyzes the relevance between blood glucose data and meal data and preferentially analyzes highly relevant data. The analysis unit can also postpone analysis of less relevant data. For example, the analysis unit analyzes the relevance between blood pressure data and exercise data and postpones analysis of less relevant data. Furthermore, the analysis unit can adjust the order of analysis according to the user's health condition. For example, the analysis unit preferentially analyzes highly relevant data when the user's health risk is high. By adjusting the order of analysis based on the relevance of health data, highly relevant data can be preferentially analyzed. Some or all of the above-described processing in the analysis unit may be performed using AI or without using AI. For example, the analysis unit can input health data into a generative AI and have the generative AI execute adjustment of the order of analysis. Specifically, the analysis unit normalizes, imputes missing values, and removes outliers from multidimensional health data received from the collection unit (e.g., blood glucose [7,1440] tensor, meal content vector [20], exercise intensity vector [5], blood pressure [14] vector, heart rate [86,400] vector) in the data preprocessing unit and inputs them into a relevance judgment module. The analysis unit uses relevance judgment modules such as calculation of correlation coefficient matrices (e.g., Pearson correlation, Spearman rank correlation) and machine learning models (e.g., feature importance estimation by random forest) to calculate relevance scores for each data pair (output example: blood glucose-meal 0.8, blood pressure-exercise 0.3). Examples of inputs to the AI model include (1) time-series data for each health indicator, (2) behavioral data vector, and (3) user attributes (age, disease history). The AI model outputs (a) relevance score matrix, and (b) recommended analysis order list (output example: blood glucose-meal→GFR-sleep→blood pressure-exercise). For example, if the relevance between blood glucose and meal is high, the analysis unit preferentially analyzes these data simultaneously to improve the accuracy of factor analysis and prediction models. Less relevant data (e.g., blood pressure-exercise) is postponed to save computational resources. If health risk is high, highly relevant data pairs are further prioritized. The AI model uses loss functions such as correlation estimation loss and analysis accuracy loss to maximize relevance judgment accuracy. Training data consists of health datasets with relevance labels by medical experts, and data augmentation such as time-series shifting and noise addition is applied. The output of the AI model is passed to the analysis pipeline control module, where rule-based branching (e.g., prioritize analysis for relevance 0.7 or higher) and threshold judgment are performed and reflected in the actual analysis flow. As a technical effect, the analysis unit, unlike conventional uniform analysis order, can accurately determine the relevance between health data and dynamically optimize the order of analysis, thereby simultaneously improving factor analysis accuracy, computational efficiency, and abnormal value detection rate. This enables risk assessment for each disease in medical settings and home health management, and personalized analysis for each user. Specific application fields include home monitoring for chronic disease patients, remote medical support, corporate health management platforms, and risk assessment systems for insurance companies.

The proposal unit is capable of estimating the user's emotion and adjusting the method of presenting proposals based on the estimated emotion of the user. For example, when the user is tense, the proposal unit provides simple and highly visible proposals. For instance, the proposal unit captures the user's facial expression with a camera and estimates the emotion using an emotion estimation algorithm. Additionally, when the user is relaxed, the proposal unit can provide detailed proposals. For example, the proposal unit records the user's voice and estimates the emotion using voice analysis technology. Furthermore, when the user is in a hurry, the proposal unit can provide proposals that focus on key points. For example, the proposal unit collects the user's biometric data (such as heart rate and skin electrical activity) with sensors and estimates the emotion using an emotion estimation algorithm. By adjusting the method of presenting proposals according to the user's emotion, more appropriate proposals can be provided. Emotion estimation is realized, for example, by using an emotion engine or generative AI with emotion estimation functions. Generative AI may include text generation AI (such as LLM) or multimodal generative AI, but is not limited to these examples. Some or all of the above-described processing in the proposal unit may be performed using AI or without using AI. For example, the proposal unit can input the user's emotion data to generative AI and have the generative AI execute the adjustment of the method of presenting proposals. Specifically, the present proposal unit simultaneously acquires multiple sensor data (e.g., facial expression images [224,224,3] tensor, voice waveform [16000] array, heart rate time series [60] vector, skin electrical activity [60] vector) to estimate the user's emotional state, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The proposal unit adopts a hybrid configuration of a multimodal Transformer and convolutional neural network (CNN) plus recurrent neural network (RNN) as the emotion estimation AI model, receiving as input examples: (1) facial expression feature vector [128], (2) voice feature vector [64], (3) heart rate variability float, and (4) skin electrical activity float. The AI model outputs (a) emotion labels (output examples: tense, relaxed, in a hurry) and (b) emotion intensity scores (output examples: continuous values from 0.0 to 1.0) from these inputs. For example, if the user is estimated to be in a tense state (emotion label: tense, intensity 0.7), the proposal unit automatically generates a simple UI that emphasizes only the key points of graphs and numerical data as the method of presenting the proposal content. In a relaxed state (emotion label: relaxed, intensity 0.2), rich proposal content including detailed graphs, explanations of time-series fluctuations, and factor analysis comments is generated. In a hurry, only the key points are presented in a bulleted list. The AI model is trained on a GPU cluster using loss functions such as cross-entropy loss and mean squared error to maximize emotion estimation accuracy. Training data includes multimodal biometric datasets with emotion labels (e.g., 10,000 samples), and data augmentation such as rotation of facial images, pitch shifting of voice, and addition of noise to heart rate is applied. The output of the AI model is passed to the proposal content generation module, and after threshold judgment (e.g., tense intensity of 0.6 or higher results in simple display) and rule-based branching (e.g., detailed display when relaxed), it is reflected in the actual proposal content UI or report generation. As a technical effect, the present proposal unit, unlike conventional uniform proposal displays, can estimate the user's emotional state with high accuracy and in real time, and dynamically optimize the method of presenting proposal content, thereby greatly improving the user's understanding, acceptance, and behavior change rate. This prevents confusion due to information overload under stress and dissatisfaction due to lack of information when relaxed, dramatically improving the user experience of health management AI. Specific application fields include home health monitoring apps, remote medical support systems, corporate health management platforms, and condition management for athletes.

The proposal unit is capable of adjusting the level of detail of proposals at the time of proposal based on the importance of the health condition. For example, the proposal unit provides detailed proposals for important health conditions. For instance, when the user's blood glucose level is high, the proposal unit provides detailed meal proposals. Additionally, for general health conditions, the proposal unit can provide simplified proposals. For example, when the user's body temperature is normal, the proposal unit provides simplified health management proposals. Furthermore, the proposal unit can adjust the level of detail of proposals according to the user's health condition. For example, when the user's health risk is high, the proposal unit provides detailed proposals. By adjusting the level of detail of proposals based on the importance of the health condition, detailed proposals can be provided for important health conditions. Some or all of the above-described processing in the proposal unit may be performed using AI or without using AI. For example, the proposal unit can input health data to generative AI and have the generative AI execute the adjustment of the level of detail of proposals. Specifically, the present proposal unit receives multidimensional health data from the analysis unit (e.g., blood glucose level [7,1440]tensor, GFR [7] vector, blood pressure [14] vector, heart rate [86400] vector, body temperature [24] vector), normalizes, imputes missing values, and removes outliers in a data preprocessing unit, and inputs the data to an importance determination module. The proposal unit uses rule-based methods (e.g., high importance if blood glucose fluctuation exceeds a threshold) or machine learning models (e.g., anomaly score estimation by random forest) as the importance determination module to calculate importance scores for each health indicator (output examples: blood glucose 0.9, GFR 0.8, body temperature 0.3). Input examples to the AI model include (1) time-series data of each health indicator, (2) user attributes (age, medical history), and (3) recent anomaly detection flags. The AI model outputs (a) importance scores (0.0-1.0) and (b) recommended proposal detail levels (output examples: detailed, standard, simplified) from these inputs. For example, if the importance score for blood glucose is high at 0.9, the proposal unit uses a recurrent neural network (RNN) or autoregressive model to estimate the contribution of meals, exercise, and sleep in detail, and generates detailed proposals including graphs and factor explanations. If the importance score for body temperature is low at 0.3, only simple moving averages or distribution summaries are presented. When health risk is high (e.g., sustained high blood glucose over the past week), detailed proposals are enforced for all indicators. The AI model is trained using loss functions such as importance classification loss and proposal accuracy loss to minimize missed important data. Training data includes health datasets labeled with importance by medical professionals, and data augmentation such as addition of anomalies and time-series shifts is applied. The output of the AI model is passed to the proposal generation module, and after rule-based branching (e.g., importance of 0.7 or higher results in detailed proposals) and threshold judgment, it is reflected in the actual proposal content. As a technical effect, the present proposal unit, unlike conventional uniform proposal processing, can accurately determine the importance of health data and concentrate computational resources on important data, thereby simultaneously improving proposal accuracy, computational efficiency, and preventing information overload for users. This greatly improves early detection of anomalies and the quality of decision support in medical settings and home health management. Specific application fields include home monitoring for chronic disease patients, remote medical support, corporate health management platforms, and risk assessment systems for insurance companies.

The proposal unit is capable of applying different proposal algorithms according to the category of health condition at the time of proposal. For example, the proposal unit applies a dedicated proposal algorithm for blood glucose level proposals. For instance, the proposal unit uses regression analysis to make proposals related to blood glucose level. Additionally, the proposal unit can apply a dedicated proposal algorithm for GFR-related proposals. For example, the proposal unit uses clustering to make proposals related to GFR. Furthermore, the proposal unit can apply a dedicated proposal algorithm for blood pressure-related proposals. For example, the proposal unit uses time-series analysis to make proposals related to blood pressure. By applying different proposal algorithms according to the category of health condition, more accurate proposals can be made. Some or all of the above-described processing in the proposal unit may be performed using AI or without using AI. For example, the proposal unit can input health data to generative AI and have the generative AI execute the application of proposal algorithms. Specifically, the present proposal unit inputs multidimensional health data received from the analysis unit (e.g., blood glucose level [7,1440] tensor, GFR [7] vector, blood pressure [14] vector, heart rate [86400] vector, body temperature [24] vector) to a data category determination module, which automatically selects the optimal proposal algorithm for each data type. For blood glucose data, time-series regression models (e.g., LSTM networks or autoregressive models) and fluctuation factor analysis algorithms (e.g., estimation of contributions from meals, exercise, and sleep) are applied; for GFR data, clustering (e.g., k-means, self-organizing maps) and anomaly detection algorithms (e.g., Isolation Forest) are applied; for blood pressure data, time-series analysis (e.g., autoregressive moving average model ARMA, recurrent neural network) and periodicity detection algorithms are applied. Input examples to the AI model include (1) blood glucose time-series tensor [7,1440], (2) GFR vector [7], (3) blood pressure vector [14], and (4) user attribute category data. The AI model outputs (a) category determination labels (output examples: blood glucose, GFR, blood pressure), (b) recommended proposal algorithm IDs (output examples: LSTM, k-means, ARMA), and (c) proposal content (e.g., blood glucose fluctuation prediction vector, GFR cluster label, blood pressure periodicity score) from these inputs. For example, blood glucose data is subjected to 24-hour prediction by LSTM, GFR data is classified into stable/unstable groups by clustering, and blood pressure data is analyzed for anomalies by periodicity analysis. The AI model is trained using loss functions such as category classification loss and proposal accuracy loss to maximize optimal algorithm selection accuracy. Training data includes health datasets labeled with proposal algorithms by medical professionals, and data augmentation such as shuffling of category labels and addition of noise is applied. The output of the AI model is passed to the proposal pipeline control module, and after rule-based branching (e.g., blood glucose uses LSTM, GFR uses k-means) and threshold judgment, it is reflected in the actual proposal flow. As a technical effect, the present proposal unit, unlike conventional uniform application of proposal algorithms, can automatically select and apply the optimal algorithm for each health data category, thereby simultaneously improving proposal accuracy, computational efficiency, and anomaly detection rate. This enables risk assessment for each disease in medical settings and home health management, as well as personalized proposals for each user. Specific application fields include home monitoring for chronic disease patients, remote medical support, corporate health management platforms, and risk assessment systems for insurance companies.

The proposal unit is capable of estimating the user's emotion and adjusting the length of proposals based on the estimated emotion of the user. For example, when the user is in a hurry, the proposal unit provides concise proposals that focus on key points. For instance, the proposal unit captures the user's facial expression with a camera and estimates the emotion using an emotion estimation algorithm. Additionally, when the user is relaxed, the proposal unit can provide detailed proposals. For example, the proposal unit records the user's voice and estimates the emotion using voice analysis technology. Furthermore, when the user is excited, the proposal unit can provide visually stimulating proposals. For example, the proposal unit collects the user's biometric data (such as heart rate and skin electrical activity) with sensors and estimates the emotion using an emotion estimation algorithm. By adjusting the length of proposals according to the user's emotion, optimal proposals can be provided to the user. Emotion estimation is realized, for example, by using an emotion engine or generative AI with emotion estimation functions. Generative AI may include text generation AI (such as LLM) or multimodal generative AI, but is not limited to these examples. Some or all of the above-described processing in the proposal unit may be performed using AI or without using AI. For example, the proposal unit can input the user's emotion data to generative AI and have the generative AI execute the adjustment of the length of proposals. Specifically, the present proposal unit simultaneously acquires multiple sensor data (e.g., facial expression images [224,224,3] tensor, voice waveform [16000] array, heart rate time series [60] vector, skin electrical activity [60] vector) to estimate the user's emotional state, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The emotion estimation AI model adopts a hybrid configuration of multimodal Transformer and CNN+RNN, receiving as input examples: (1) facial expression feature vector [128], (2) voice feature vector [64], (3) heart rate variability float, and (4) skin electrical activity float. The AI model outputs (a) emotion labels (output examples: in a hurry, relaxed, excited) and (b) emotion intensity scores (output examples: continuous values from 0.0 to 1.0) from these inputs. For example, if the user is estimated to be in a hurry (emotion label: in a hurry, intensity 0.8), the proposal unit automatically shortens the length of the proposal content and presents only the key points in a bulleted list or graph. In a relaxed state (emotion label: relaxed, intensity 0.2), a long report including detailed graphs, explanations of time-series fluctuations, and factor analysis comments is generated. In an excited state, visually stimulating proposal content utilizing colors and animations is generated. The AI model is trained on a GPU cluster using loss functions such as cross-entropy loss and mean squared error to maximize emotion estimation accuracy and proposal length optimization accuracy. Training data includes multimodal biometric datasets with emotion labels (e.g., 10,000 samples), and data augmentation such as rotation of facial images, pitch shifting of voice, and addition of noise to heart rate is applied. The output of the AI model is passed to the proposal content generation module, and after threshold judgment (e.g., in a hurry intensity of 0.7 or higher results in shortened display) and rule-based branching (e.g., detailed display when relaxed), it is reflected in the actual proposal content UI or report generation. As a technical effect, the present proposal unit, unlike conventional uniform proposal displays, can estimate the user's emotional state with high accuracy and in real time, and dynamically optimize the length of proposal content, thereby greatly improving the user's understanding, acceptance, and behavior change rate. This prevents confusion due to information overload and dissatisfaction due to lack of information, dramatically improving the user experience of health management AI. Specific application fields include home health monitoring apps, remote medical support systems, corporate health management platforms, and condition management for athletes.

The proposal unit is capable of determining the priority of proposals at the time of proposal based on the timing of health data collection. For example, the proposal unit makes proposals based on recently collected data. For instance, the proposal unit makes proposals based on the latest blood glucose level data. Additionally, the proposal unit can make proposals based on current data while referring to past data. For example, the proposal unit makes proposals based on current GFR data while referring to GFR data from the past year. Furthermore, the proposal unit can adjust the priority of proposals according to the user's health condition. For example, when the user's health risk is high, the proposal unit makes proposals based on the latest data. By determining the priority of proposals based on the timing of health data collection, proposals based on the latest data can be made. Some or all of the above-described processing in the proposal unit may be performed using AI or without using AI. For example, the proposal unit can input health data to generative AI and have the generative AI execute the determination of proposal priority. Specifically, the present proposal unit obtains multidimensional health data (e.g., blood glucose level [365,1440] tensor, GFR [365] vector, blood pressure [365, 2]vector) received from the analysis unit from the database, and inputs it together with data collection time information to a priority determination module. The proposal unit uses rule-based methods (e.g., latest data has high priority) or machine learning models (e.g., anomaly score estimation by random forest) as the priority determination module to calculate proposal priority scores for each data (output examples: latest blood glucose level 0.9, GFR from one week ago 0.5). Input examples to the AI model include (1) time-series data of each health indicator, (2) data collection time, (3) user attributes (age, medical history), and (4) recent anomaly detection flags. The AI model outputs (a) proposal priority scores (0.0-1.0) and (b) recommended proposal order lists (output examples: latest blood glucose level→latest GFR→past blood pressure) from these inputs. For example, when health risk is high (e.g., sustained high blood glucose over the past week), the priority of the latest data is further increased and proposals are made accordingly. The AI model is trained using loss functions such as priority classification loss and proposal accuracy loss to minimize missed important data. Training data includes health datasets labeled with priority by medical professionals, and data augmentation such as time-series shifts and addition of anomalies is applied. The output of the AI model is passed to the proposal pipeline control module, and after rule-based branching (e.g., latest data is always top priority) and threshold judgment, it is reflected in the actual proposal flow. As a technical effect, the present proposal unit, unlike conventional uniform proposal order, can dynamically optimize proposal priority according to the timing of health data collection and user status, thereby simultaneously achieving early detection of anomalies, rapid response, and efficient use of computational resources. This enables real-time risk management and personalized proposals for each user in medical settings and home health management. Specific application fields include home monitoring for chronic disease patients, remote medical support, corporate health management platforms, and risk assessment systems for insurance companies.

The proposal unit is capable of adjusting the order of proposals at the time of proposal based on the relevance of health data. For example, the proposal unit makes proposals based on highly relevant data. For instance, the proposal unit analyzes the relevance between blood glucose level data and meal data and makes proposals based on highly relevant data. Additionally, the proposal unit can postpone proposals based on less relevant data. For example, the proposal unit analyzes the relevance between blood pressure data and exercise data and postpones proposals based on less relevant data. Furthermore, the proposal unit can adjust the order of proposals according to the user's health condition. For example, when the user's health risk is high, the proposal unit makes proposals based on highly relevant data. By adjusting the order of proposals based on the relevance of health data, proposals based on highly relevant data can be made. Some or all of the above-described processing in the proposal unit may be performed using AI or without using AI. For example, the proposal unit can input health data to generative AI and have the generative AI execute the adjustment of proposal order. Specifically, the present proposal unit receives multidimensional health data from the analysis unit (e.g., blood glucose level [7,1440] tensor, meal content vector [20], exercise intensity vector [5], blood pressure [14] vector, heart rate [86400] vector), normalizes, imputes missing values, and removes outliers in a data preprocessing unit, and inputs the data to a relevance determination module. The proposal unit uses correlation coefficient matrix calculation (e.g., Pearson correlation, Spearman rank correlation) or machine learning models (e.g., feature importance estimation by random forest) as the relevance determination module to calculate relevance scores for each data pair (output examples: blood glucose-meal 0.8, blood pressure-exercise 0.3). Input examples to the AI model include (1) time-series data of each health indicator, (2) behavior data vector, and (3) user attributes (age, medical history). The AI model outputs (a) relevance score matrix and (b) recommended proposal order list (output examples: blood glucose-meal→GFR-sleep→blood pressure-exercise) from these inputs. For example, when the relevance between blood glucose and meal is high, the proposal unit prioritizes simultaneous analysis of these data to improve the accuracy of factor analysis and prediction models. Less relevant data (e.g., blood pressure-exercise) is postponed to save computational resources. When health risk is high, highly relevant data pairs are further prioritized. The AI model is trained using loss functions such as correlation estimation loss and proposal accuracy loss to maximize relevance determination accuracy. Training data includes health datasets labeled with relevance by medical professionals, and data augmentation such as time-series shifts and addition of noise is applied. The output of the AI model is passed to the proposal pipeline control module, and after rule-based branching (e.g., relevance of 0.7 or higher results in prioritized proposals) and threshold judgment, it is reflected in the actual proposal flow. As a technical effect, the present proposal unit, unlike conventional uniform proposal order, can accurately determine the relevance between health data and dynamically optimize proposal order, thereby simultaneously improving factor analysis accuracy, computational efficiency, and anomaly detection rate. This enables risk assessment for each disease and personalized proposals for each user in medical settings and home health management. Specific application fields include home monitoring for chronic disease patients, remote medical support, corporate health management platforms, and risk assessment systems for insurance companies.

The provision unit is capable of estimating the user's emotion and adjusting the method of provision based on the estimated emotion of the user. For example, when the user is tense, the provision unit provides information in a simple and highly visible manner. For instance, the provision unit captures the user's facial expression with a camera and estimates the emotion using an emotion estimation algorithm. Additionally, when the user is relaxed, the provision unit can provide information in a manner that includes detailed information. For example, the provision unit records the user's voice and estimates the emotion using voice analysis technology. Furthermore, when the user is in a hurry, the provision unit can provide information focusing on key points. For example, the provision unit collects the user's biometric data (such as heart rate and skin electrical activity) with sensors and estimates the emotion using an emotion estimation algorithm. By adjusting the method of provision according to the user's emotion, more appropriate information provision becomes possible. Emotion estimation is realized, for example, by using an emotion engine or generative AI with emotion estimation functions. Generative AI may include text generation AI (such as LLM) or multimodal generative AI, but is not limited to these examples. Some or all of the above-described processing in the provision unit may be performed using AI or without using AI. For example, the provision unit can input the user's emotion data to generative AI and have the generative AI execute the adjustment of the method of provision. Specifically, the present provision unit simultaneously acquires multiple sensor data (e.g., facial expression images [224,224,3] tensor, voice waveform [16000] array, heart rate time series [60] vector, skin electrical activity [60] vector) to estimate the user's emotional state with high accuracy, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The provision unit adopts a hybrid configuration of a multimodal Transformer and convolutional neural network (CNN) plus recurrent neural network (RNN) as the emotion estimation AI model, receiving as input examples: (1) facial expression feature vector [128], (2) voice feature vector [64], (3) heart rate variability float, and (4) skin electrical activity float. The AI model outputs (a) emotion labels (output examples: tense, relaxed, in a hurry) and (b) emotion intensity scores (output examples: continuous values from 0.0 to 1.0) from these inputs. For example, if the user is estimated to be in a tense state (emotion label: tense, intensity 0.7), the provision unit automatically generates a simple UI that emphasizes only the key points of graphs and numerical data as the method of presenting the provision content. In a relaxed state (emotion label: relaxed, intensity 0.2), rich information provision including detailed graphs, explanations of time-series fluctuations, and factor analysis comments is generated. In a hurry, only the key points are presented in a bulleted list. The AI model is trained on a GPU cluster using loss functions such as cross-entropy loss and mean squared error to maximize emotion estimation accuracy. Training data includes multimodal biometric datasets with emotion labels (e.g., 10,000 samples), and data augmentation such as rotation of facial images, pitch shifting of voice, and addition of noise to heart rate is applied. The output of the AI model is passed to the provision method generation module, and after threshold judgment (e.g., tense intensity of 0.6 or higher results in simple display) and rule-based branching (e.g., detailed display when relaxed), it is reflected in actual notifications, emails, or in-app message generation. As a technical effect, the present provision unit, unlike conventional uniform information provision, can estimate the user's emotional state with high accuracy and in real time, and dynamically optimize the method of provision, thereby greatly improving the user's understanding, acceptance, and behavior change rate. This prevents confusion due to information overload under stress and dissatisfaction due to lack of information when relaxed, dramatically improving the user experience of health management AI. Specific application fields include home health monitoring apps, remote medical support systems, corporate health management platforms, and condition management for athletes.

The provision unit is capable of selecting a method of provision at the time of provision based on the user's past behavioral history. For example, the provision unit preferentially selects a method of provision that the user has preferred to use in the past. For instance, the provision unit analyzes the user's past behavioral history to identify the preferred method of provision. Additionally, the provision unit can select the optimal timing of provision based on the user's past behavioral history. For example, the provision unit analyzes the user's past behavioral history to identify the optimal timing of provision. Furthermore, the provision unit can select the optimal format of provision based on the user's past behavioral history. For example, the provision unit analyzes the user's past behavioral history to identify the optimal format of provision. By referring to the user's past behavioral history, the optimal method of provision can be selected. Some or all of the above-described processing in the provision unit may be performed using AI or without using AI. For example, the provision unit can input the user's behavioral history data to generative AI and have the generative AI execute the selection of the method of provision. Specifically, the present provision unit obtains behavioral log data for each user, such as notification opening history, email viewing history, and in-app message reaction history (e.g., notification opening time list [100], email viewing flag array [50], in-app message read flag [30], reaction score float for each provision method) from the database and inputs it to a behavioral history analysis AI model. The provision unit first uses a time-series analysis module (e.g., LSTM network) to extract which provision method, timing, and format the user responded to most. Input examples to the AI model include (1) notification opening history vector [100], (2) email viewing history vector [50], (3) in-app message read history [30], and (4) reaction score float for each provision method. The AI model outputs (a) recommended provision method ID (output examples: push notification ID1, email ID2, in-app message ID3), (b) recommended provision timing (output examples: 8 a.m., 8 p.m., immediate), and (c) recommended provision format (output examples: key points only, with detailed explanation) from these inputs. For example, if the user has shown a high response rate to push notifications at 8 a.m. in the past, the provision unit sends notifications at that timing. The AI model is trained using loss functions such as response prediction loss and classification loss to maximize optimal provision method selection accuracy. Training data includes behavioral history datasets for many users, and data augmentation such as time-series shuffling of history and addition of noise is applied. The output of the AI model is passed to the provision scheduler, and after rule-based branching (e.g., response rate of 70% or higher in the past results in prioritizing the same method) and threshold judgment, it is reflected in actual notification, email, or in-app message generation. As a technical effect, the present provision unit, unlike conventional uniform information provision, can analyze the user's past behavioral history with high accuracy and dynamically select the optimal method, timing, and format of provision, thereby improving user response rate, information transmission efficiency, and continuity of health management. Specific application fields include home health management apps, remote medical support systems, corporate health management platforms, and risk notification systems for insurance companies.

The provision unit is capable of estimating the user's emotion and determining the priority of provision based on the estimated emotion of the user. For example, when the user is feeling stressed, the provision unit prioritizes information related to stress reduction. For instance, the provision unit captures the user's facial expression with a camera and estimates the emotion using an emotion estimation algorithm. Additionally, when the user is relaxed, the provision unit can provide overall health information in a balanced manner. For example, the provision unit records the user's voice and estimates the emotion using voice analysis technology. Furthermore, when the user is in a hurry, the provision unit can prioritize only important information. For example, the provision unit collects the user's biometric data (such as heart rate and skin electrical activity) with sensors and estimates the emotion using an emotion estimation algorithm. By determining the priority of provision according to the user's emotion, important information can be provided preferentially. Emotion estimation is realized, for example, by using an emotion engine or generative AI with emotion estimation functions. Generative AI may include text generation AI (such as LLM) or multimodal generative AI, but is not limited to these examples. Some or all of the above-described processing in the provision unit may be performed using AI or without using AI. For example, the provision unit can input the user's emotion data to generative AI and have the generative AI execute the determination of provision priority. Specifically, the present provision unit simultaneously acquires multiple sensor data (e.g., facial expression images [224,224,3] tensor, voice waveform [16000] array, heart rate time series [60] vector, skin electrical activity [60] vector) to estimate the user's emotional state, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The provision unit adopts a hybrid configuration of a multimodal Transformer and convolutional neural network (CNN) plus recurrent neural network (RNN) as the emotion estimation AI model, receiving as input examples: (1) facial expression feature vector [128], (2) voice feature vector [64], (3) heart rate variability float, and (4) skin electrical activity float. The AI model outputs (a) emotion labels (output examples: stress, relaxed, in a hurry), (b) emotion intensity scores (output examples: continuous values from 0.0 to 1.0), and (c) recommended provision priority list (output examples: stress reduction information prioritized during stress, balanced overall information during relaxation, important information only when in a hurry) from these inputs. For example, if the user is estimated to be in a stress state (emotion label: stress, intensity 0.8), the provision unit prioritizes information related to stress reduction (e.g., relaxation methods, stress relief exercises). In a relaxed state, overall health information is provided in a balanced manner. When in a hurry, only important information (e.g., medication instructions, emergency contacts) is prioritized. The AI model is trained using loss functions such as cross-entropy loss and priority determination loss to maximize emotion estimation accuracy and priority determination accuracy. Training data includes multimodal biometric datasets with emotion labels and information provision datasets with priority labels, and data augmentation such as rotation of facial images, addition of noise to voice, and variation of heart rate is applied. The output of the AI model is passed to the provision priority control module, and after rule-based branching (e.g., stress intensity of 0.7 or higher results in prioritizing stress-related information) and threshold judgment, it is reflected in actual notification, email, or in-app message generation. As a technical effect, the present provision unit, unlike conventional uniform information provision, can estimate the user's emotional state with high accuracy and dynamically optimize the priority of information provision, thereby preventing missed important health indicators and minimizing user burden. Specific application fields include stress management systems, home health monitoring, remote medical support, and corporate health management platforms.

The provision unit is capable of selecting an optimal method of provision at the time of provision in consideration of the user's device information. For example, when the user is using a smartphone, the provision unit selects a method of provision optimized for the screen size. For instance, the provision unit sends notifications optimized for the smartphone screen size. Additionally, when the user is using a tablet, the provision unit can select a method of provision optimized for the larger screen. For example, the provision unit sends emails optimized for the tablet screen size. Furthermore, when the user is using a smartwatch, the provision unit can select a concise and highly visible method of provision. For example, the provision unit sends in-app messages optimized for the smartwatch screen size. By considering the user's device information, the optimal method of provision can be selected. Some or all of the above-described processing in the provision unit may be performed using AI or without using AI. For example, the provision unit can input the user's device information to generative AI and have the generative AI execute the selection of the method of provision. Specifically, the present provision unit obtains the user's device information (e.g., device type ID, screen size float, OS version, notification permission settings, communication environment label) in real time and inputs it to a device optimization AI model. The provision unit uses fully connected neural networks or decision tree models as the device optimization AI model, receiving as input examples: (1) device type ID (e.g., smartphone ID1, tablet ID2, smartwatch ID3), (2) screen size float, (3) OS version, (4) notification permission setting binary, and (5) communication environment label (e.g., Wi-Fi, 4G, 5G). The AI model outputs (a) recommended provision method ID (output examples: push notification ID1, email ID2, in-app message ID3), (b) recommended UI layout (output examples: simple, detailed, graph-centric), and (c) recommended data amount (output examples: text only, with images, with video) from these inputs. For example, when the user is using a smartwatch, the provision unit immediately sends only simple text notifications. For tablets, detailed emails including graphs and images are sent. The AI model is trained using loss functions such as provision method selection loss and UI optimization loss to maximize optimal provision method selection accuracy. Training data includes user response datasets under various device environments, and data augmentation such as shuffling of device types and addition of noise to communication environments is applied. The output of the AI model is passed to the provision scheduler, and after rule-based branching (e.g., screen size less than 3 inches results in simple UI) and threshold judgment, it is reflected in actual notification, email, or in-app message generation. As a technical effect, the present provision unit, unlike conventional uniform information provision, can analyze the user's device characteristics with high accuracy and dynamically select the optimal method of provision, UI, and data amount, thereby improving user information receptivity, transmission efficiency, and satisfaction. Specific application fields include home health management apps, remote medical support systems, corporate health management platforms, and risk notification systems for insurance companies.

The system according to the embodiment is not limited to the examples described above and can be variously modified as follows, for example. Specifically, the present system can flexibly change the types of AI models, data flow, user interface, communication methods, sensor configuration, and database structure in each module for health data collection, analysis, proposal, and provision. The system can realize not only batch processing by a single AI model but also distributed processing configurations that link multiple AI models (e.g., cooperative operation of emotion estimation AI and behavior recognition AI, linkage of time-series prediction AI and anomaly detection AI), as well as cloud-edge cooperative distributed learning configurations (e.g., primary feature extraction on edge devices plus detailed analysis in the cloud). Furthermore, the system can integrate various data sources other than health data, such as lifestyle data, environmental data, social media data, and wearable device data, and perform comprehensive health risk assessment and personalized proposal generation using multimodal AI. In addition, the system can utilize metadata such as user attributes (age, gender, medical history, genetic information), family history, occupation, and living environment to implement individually optimized health management algorithms. The learning methods for AI models can also be flexibly configured according to data privacy and security requirements, adopting various methods such as supervised learning, semi-supervised learning, transfer learning, and federated learning. As a technical effect, the present system, unlike conventional health management systems with single functions and fixed configurations, can realize flexible and highly extensible system design according to user characteristics, usage environment, and diversity of data sources, thereby improving the accuracy of personalized health management, system operation efficiency, anomaly detection rate, and optimization of user experience. Specific application fields include home health monitoring, remote medical support, corporate health management platforms, risk assessment systems for insurance companies, performance management for athletes, and care support systems.

The collection unit is capable of adjusting the timing of data collection when collecting the user's health data by considering the user's lifestyle and daily behavior patterns. For example, when the user has a habit of jogging every morning, the collection unit collects blood glucose level and heart rate data after jogging. Additionally, the collection unit can collect blood pressure and body temperature data during the user's nighttime relaxation period. Furthermore, the collection unit can collect postprandial blood glucose level data according to the user's meal times. By collecting health data at optimal timing based on the user's lifestyle, more accurate data can be obtained. Specifically, the present collection unit automatically acquires the user's lifestyle data (e.g., time-series array of jogging start and end times [7], meal start time list [21], sleep and wake time vector [7], relaxation time flag [24]) from wearable devices or smartphone apps and inputs these data to a lifestyle analysis AI model. The collection unit uses time-series clustering algorithms (e.g., Dynamic Time Warping clustering) or recurrent neural networks (RNN) as the lifestyle analysis AI model to extract the user's behavior patterns with high accuracy. Input examples to the AI model include (1) jogging time array [7], (2) meal time list [21], (3) relaxation time flag [24], and (4) user attributes (age, occupation, lifestyle rhythm). The AI model outputs (a) behavior pattern labels (output examples: morning type, night type, exercise habit), (b) recommended data collection timing list (output examples: 30 minutes after jogging, 1 hour after meals, before bedtime), and (c) collection priority score (0.0-1.0). For example, if the user jogs at 6 a.m. every morning, the collection unit automatically activates blood glucose and heart rate sensors at 6:30 a.m. to acquire data. During nighttime relaxation time (e.g., 9-10 p.m.), blood pressure and body temperature sensors are preferentially operated. If meal times are 12 p.m. and 7 p.m., blood glucose sensors are operated 1 hour after each meal. The AI model is trained using loss functions such as behavior pattern classification loss and collection timing optimization loss to maximize the estimation accuracy of optimal data collection timing. Training data includes time-series sets of behavior logs and health data from users with diverse lifestyles, and data augmentation such as random shifts of behavior times and addition of lifestyle rhythm variations is applied. The output of the AI model is passed to the collection scheduler, and after rule-based branching (e.g., always collect heart rate and blood glucose after exercise) and threshold judgment (e.g., relaxation time intensity of 0.7 or higher results in blood pressure collection), it is reflected in actual sensor control and data collection planning. As a technical effect, the present collection unit, unlike conventional fixed-time and uniform data collection, can analyze each user's lifestyle and behavior patterns with high accuracy and collect health data at optimal timing, thereby greatly improving the reliability and usefulness of data by not missing behavioral changes or physiological fluctuations. This enables personalized health monitoring for each user in medical settings, home health management, sports performance management, and lifestyle disease prevention programs.

The collection unit is capable of adjusting the frequency of data collection when collecting the user's health data by considering the user's emotional state. For example, when the user is feeling stressed, the collection unit can reduce the frequency of data collection until the stress is alleviated. Additionally, when the user is relaxed, the collection unit can increase the frequency of data collection. Furthermore, when the user is in a hurry, the collection unit can adjust to collect data in a short period of time. By adjusting the frequency of data collection according to the user's emotional state, accurate data can be collected without burdening the user. Specifically, the present collection unit simultaneously acquires multiple biometric signals (e.g., facial expression images [224,224,3], voice waveform [16000], heart rate [60], skin electrical activity [60]) to estimate the user's emotional state, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The emotion estimation AI model adopts a hybrid configuration of multimodal Transformer and CNN+RNN, receiving as input examples: (1) facial expression feature vector [128], (2) voice feature vector [64], (3) heart rate variability float, and (4) skin electrical activity float. The AI model outputs (a) emotion labels (output examples: stress, relaxed, in a hurry), (b) emotion intensity scores (0.0-1.0), and (c) recommended collection frequency (output examples: once per day, three times per day, every 10 minutes) from these inputs. For example, if the user is estimated to be in a stress state (emotion label: stress, intensity 0.8), the collection unit reduces the data collection frequency to once per day to reduce user burden. In a relaxed state (emotion label: relaxed, intensity 0.2), data is collected frequently, such as three times per day or every 10 minutes. When in a hurry, only the minimum necessary data is collected in a short period of time. The AI model is trained using loss functions such as cross-entropy loss and frequency optimization loss to maximize emotion estimation accuracy and collection frequency optimization accuracy. Training data includes multimodal biometric datasets with emotion labels and health datasets with collection frequency labels, and data augmentation such as rotation of facial images, addition of noise to voice, and variation of heart rate is applied. The output of the AI model is passed to the collection frequency control module, and after rule-based branching (e.g., stress intensity of 0.7 or higher results in frequency of once per day) and threshold judgment, it is reflected in the actual data collection scheduler. As a technical effect, the present collection unit, unlike conventional uniform data collection frequency settings, can estimate the user's emotional state with high accuracy and dynamically optimize the collection frequency, thereby minimizing user burden while accurately acquiring necessary health data. This enables the coexistence of user experience and data quality in stress management systems, home health monitoring, remote medical support, and corporate health management platforms.

The analysis unit is capable of identifying abnormal values by comparing collected data with the user's past health data during analysis. For example, the analysis unit compares the user's blood glucose level data from the past year with current blood glucose level data to identify abnormal fluctuations. Additionally, the analysis unit can compare the user's past GFR data with current GFR data to identify abnormalities in renal function. Furthermore, the analysis unit can compare the user's past blood pressure data with current blood pressure data to identify abnormalities in blood pressure. By comparing with the user's past health data, abnormal values can be detected early and appropriate measures can be taken. Specifically, the present analysis unit obtains multidimensional health data (e.g., blood glucose level [365,1440] tensor, GFR [365] vector, blood pressure [365,2] vector) received from the collection unit from the database and inputs it to an anomaly detection AI model. The anomaly detection AI model uses time-series anomaly detection algorithms (e.g., LSTM Autoencoder, Isolation Forest, One-Class SVM) and statistical anomaly detection methods (e.g., moving average plus standard deviation threshold judgment) in combination. Input examples to the AI model include (1) blood glucose time-series tensor [365,1440], (2) GFR vector [365], (3) blood pressure vector [365,2], (4) user attributes (age, medical history), and (5) recent anomaly detection flags. The AI model outputs (a) anomaly scores (0.0-1.0), (b) anomaly detection labels (output examples: normal, abnormal, caution), and (c) anomaly location index list (e.g., blood glucose: 2024 May 1 8:00, GFR: 2024 Apr. 15, blood pressure: 2024 May 3 20:00) from these inputs. For example, if the anomaly score for blood glucose is high at 0.9, the analysis unit identifies the blood glucose at that time as an abnormal value and generates an alert. For GFR and blood pressure, sudden changes or deviations from reference values are detected by comparison with past data. The AI model is trained using loss functions such as anomaly detection loss and false detection suppression loss to maximize anomaly detection accuracy and reduce false alarm rates. Training data includes health datasets labeled with anomalies by medical professionals, and data augmentation such as addition of anomalies and time-series shifts is applied. The output of the AI model is passed to the anomaly alert generation module, and after rule-based branching (e.g., anomaly score of 0.8 or higher results in immediate notification) and threshold judgment, it is reflected in actual notification or report generation. As a technical effect, the present analysis unit, unlike conventional simple threshold judgment or manual anomaly detection, can compare each user's past and current data in a high-dimensional time-series space and detect nonlinear abnormal patterns with high accuracy, enabling early detection of anomalies, reduction of false alarms, and rapid response. This enables personalized anomaly detection for each user in home monitoring for chronic disease patients, remote medical support, corporate health management platforms, and risk assessment systems for insurance companies.

The proposal unit is capable of considering the user's preferences and allergy information when proposing meals or actions based on the user's health condition. For example, when the user has an allergy to a specific food ingredient, the proposal unit proposes meals that do not contain that ingredient. Additionally, the proposal unit can propose recipes using ingredients preferred by the user. Furthermore, the proposal unit can propose healthy meal plans based on the user's dietary preferences. By considering the user's preferences and allergy information, proposals can be made that allow the user to continue healthy eating without difficulty. Specifically, the present proposal unit integrates the user's preference data (e.g., preferred ingredient list [20], disliked ingredient list [10], allergy ingredient list [5], past meal history [30]), health condition data (e.g., blood glucose level [7,1440], GFR [7], blood pressure [14]), and user attributes (age, gender, medical history), and inputs them to a personalized meal proposal AI model. The AI model uses recipe recommendation algorithms (e.g., multimodal Transformer, graph neural network) and rule-based filters (e.g., exclusion of allergy ingredients, preference prioritization) in combination. Input examples to the AI model include (1) preferred ingredient vector [20], (2) allergy ingredient vector [5], (3) health indicator vector [10], and (4) past meal history vector [30]. The AI model outputs (a) recommended recipe ID list (output examples: recipe ID 123, 456, 789), (b) recommended reason text (e.g., no allergy ingredients used, preferred ingredients used), and (c) health score (0.0-1.0) from these inputs. For example, if the user has an egg allergy, only recipes without eggs are proposed, and recipes using preferred ingredients (e.g., chicken, tomato) are presented preferentially. The AI model is trained using loss functions such as preference matching loss and health score optimization loss to maximize preference and allergy consideration accuracy and health maintenance effect. Training data includes meal history datasets with user preferences, allergies, and health indicators, and data augmentation such as shuffling of preference labels and addition of allergy ingredients is applied. The output of the AI model is passed to the proposal generation module, and after rule-based branching (e.g., allergy ingredients are always excluded) and threshold judgment, it is reflected in the actual proposal content. As a technical effect, the present proposal unit, unlike conventional uniform meal proposals, can analyze each user's preferences, allergies, and health condition with high accuracy and realize personalized meal proposals that can be continued without difficulty, thereby simultaneously achieving health maintenance, disease prevention, and improvement of user satisfaction. This can be widely used in lifestyle disease prevention programs, home health management, corporate health management support, and risk reduction measures for insurance companies.

The provision unit is capable of adjusting the method of provision when providing proposal content to the user by considering the user's emotional state. For example, when the user is feeling stressed, the provision unit provides proposal content in a simple and highly visible manner. Additionally, when the user is relaxed, the provision unit can provide proposal content in a manner that includes detailed information. Furthermore, when the user is in a hurry, the provision unit can provide proposal content focusing on key points. By adjusting the method of provision according to the user's emotional state, optimal information provision for the user becomes possible. Specifically, the present provision unit simultaneously acquires multiple sensor data (e.g., facial expression images [224,224,3] tensor, voice waveform array, heart rate time series [60] vector, skin electrical activity [60] vector) to estimate the user's emotional state, normalizes, removes noise, and extracts features (e.g., facial expression feature vector [128], voice spectral feature [64], heart rate variability index float, skin electrical activity change float) in a multimodal preprocessing unit, and inputs them to an emotion estimation AI model. The emotion estimation AI model adopts a hybrid configuration of multimodal Transformer and CNN+RNN, receiving as input examples: (1) facial expression feature vector [128], (2) voice feature vector [64], (3) heart rate variability float, and (4) skin electrical activity float. The AI model outputs (a) emotion labels (output examples: stress, relaxed, in a hurry), (b) emotion intensity scores (0.0-1.0), and (c) recommended provision method ID (output examples: simple UI, detailed UI, bulleted key points) from these inputs. For example, if the user is estimated to be in a stress state (emotion label: stress, intensity 0.8), the provision unit generates a notification with a simple UI that emphasizes only the key points. In a relaxed state, rich information provision including detailed graphs and explanations is provided. When in a hurry, only the key points are presented in a bulleted list. The AI model is trained using loss functions such as cross-entropy loss and UI optimization loss to maximize emotion estimation accuracy and provision method optimization accuracy. Training data includes multimodal biometric datasets with emotion labels and UI preference label datasets, and data augmentation such as rotation of facial images, addition of noise to voice, and variation of heart rate is applied. The output of the AI model is passed to the provision method generation module, and after rule-based branching (e.g., stress intensity of 0.7 or higher results in simple UI) and threshold judgment, it is reflected in actual notification, email, or in-app message generation. As a technical effect, the present provision unit, unlike conventional uniform information provision, can estimate the user's emotional state with high accuracy and dynamically optimize the method of provision, thereby greatly improving the user's understanding, acceptance, and behavior change rate. This prevents confusion due to information overload under stress and dissatisfaction due to lack of information when relaxed, dramatically improving the user experience of health management AI.

The proposal unit is capable of considering the user's emotion data when receiving user feedback and improving the proposal content. For example, when the user shows a positive emotion toward a proposal, the proposal unit maintains the proposal content. Additionally, when the user shows a negative emotion toward a proposal, the proposal unit can improve the proposal content. Furthermore, the proposal unit can analyze the user's emotion data to identify points for improvement in the proposal content. By improving the proposal content in consideration of the user's emotion data, more appropriate proposals can be provided to the user. Specifically, the present proposal unit integrates user feedback data (e.g., reaction button selection history for proposal content [30], text comments, facial expression images [224,224,3], voice feedback [16000]), emotion data (e.g., emotion label, emotion intensity score), and past proposal history, and inputs them to a proposal improvement AI model. The AI model combines natural language processing algorithms (e.g., Transformer-based emotion classifier), image emotion analysis models (e.g., CNN), and voice emotion analysis models (e.g., RNN) to estimate the user's emotional response with high accuracy. Input examples to the AI model include (1) reaction history vector [30], (2) text comments (up to 512 tokens), (3) facial expression feature vector [128], (4) voice feature vector [64], and (5) past proposal content ID. The AI model outputs (a) emotion response label (output examples: positive, negative, neutral), (b) improvement necessity score (0.0-1.0), and (c) recommended improvement action (output examples: maintain content, modify content, add detailed explanation) from these inputs. For example, when the user shows a negative emotion, the AI model outputs a high improvement necessity score and recommends modification of the proposal content or addition of explanations. For positive emotions, the content is maintained. The AI model is trained using loss functions such as emotion classification loss and improvement action optimization loss to maximize emotion response estimation accuracy and improvement proposal accuracy. Training data includes datasets with user feedback, emotion labels, and improvement history, and data augmentation such as paraphrase generation of comments and rotation of facial expression images is applied. The output of the AI model is passed to the proposal improvement module, and after rule-based branching (e.g., improvement necessity of 0.7 or higher results in content modification) and threshold judgment, it is reflected in the actual proposal content. As a technical effect, the present proposal unit, unlike conventional uniform proposal content generation, can analyze the user's emotional response with high accuracy and dynamically improve the proposal content, thereby greatly improving user satisfaction, continuity, and health behavior change rate. This enables personalized proposal improvement for each user in home health management apps, remote medical support, and corporate health management platforms.

The collection unit is capable of adjusting the timing of data collection when collecting the user's health data by considering the user's geographic location information. For example, when the user is at a high altitude, the collection unit preferentially collects data related to oxygen concentration. Additionally, when the user is in an urban area, the collection unit can preferentially collect data related to environmental pollution. Furthermore, when the user is at a sports facility, the collection unit can preferentially collect data related to exercise. By considering geographic location information, highly relevant data can be collected. Specifically, the present collection unit obtains the user's location information (e.g., GPS coordinates [2], altitude float, facility ID, surrounding environment data vector [10]) in real time and inputs it to a geographic information analysis AI model. The geographic information analysis AI model uses location information embedding plus fully connected networks or decision tree models to classify the characteristics of the user's current location (e.g., high altitude, urban area, sports facility, residential area) and estimate the types of health data and collection timing that are highly relevant. Input examples to the AI model include (1) GPS coordinates [2], (2) altitude float, (3) facility ID, and (4) surrounding environment data vector [10]. The AI model outputs (a) current location category (output examples: high altitude, urban area, sports facility), (b) recommended data types for collection (output examples: oxygen concentration and heart rate at high altitude, environmental pollution and respiratory function in urban areas, exercise intensity and calorie consumption at sports facilities), and (c) recommended collection timing (output examples: immediately after arrival at high altitude, during stay in urban area, immediately after exercise) from these inputs. For example, when the user is at a high altitude of 2000 m, the collection unit preferentially collects oxygen concentration and heart rate data immediately after arrival. In urban areas, environmental pollution indicators and respiratory function data are collected during the stay. At sports facilities, data such as exercise intensity and calorie consumption are collected immediately after exercise. The AI model is trained using loss functions such as geographic information classification loss and collection optimization loss to maximize geographic information classification accuracy and collection timing optimization accuracy. Training data includes health datasets from various geographic environments, and data augmentation such as addition of noise to location information and variation of environmental data is applied. The output of the AI model is passed to the collection scheduler, and after rule-based branching (e.g., oxygen concentration is prioritized at high altitude, environmental pollution is prioritized in urban areas) and threshold judgment, it is reflected in actual data collection planning. As a technical effect, the present collection unit, unlike conventional uniform data collection, can analyze the user's geographic location information with high accuracy and collect highly relevant health data at optimal timing according to the environment, thereby enabling early detection of health risks due to environmental factors and personalized health management. This is useful for health monitoring of high-altitude climbers, environmental risk management for urban residents, performance management for sports facility users, and remote medical support.

The analysis unit is capable of adjusting the level of detail of analysis when analyzing collected data based on the user's living environment and occupation. For example, when the user is engaged in desk work, the analysis unit analyzes health risks associated with prolonged sitting in detail. Additionally, when the user is engaged in physical labor, the analysis unit can analyze data related to physical load in detail. Furthermore, when the user is working night shifts, the analysis unit can analyze sleep patterns and stress levels in detail. By adjusting the level of detail of analysis based on the user's living environment and occupation, more appropriate analysis results can be provided. Specifically, the present analysis unit integrates the user's living environment data (e.g., occupation category ID, work time vector [7], sitting time [24], physical activity amount [24], sleep time [7]), health data (e.g., blood glucose level [7,1440], GFR [7], blood pressure [14], heart rate [86400], body temperature [24]), and inputs them to a living environment adaptive analysis AI model. The AI model automatically selects the optimal analysis algorithm for each occupation and living environment (e.g., sitting time risk analysis for desk work, activity variation analysis for physical labor, sleep pattern anomaly detection for night shifts) and determines the level of detail of analysis (output examples: detailed, standard, simplified). Input examples to the AI model include (1) occupation category ID, (2) work time vector [7], (3) sitting time [24], (4) physical activity amount [24], (5) sleep time [7], and (6) health indicator vector [10]. The AI model outputs (a) recommended analysis algorithm ID (output examples: sitting risk analysis, activity analysis, sleep anomaly detection), (b) recommended analysis detail level (output examples: detailed, standard, simplified), and (c) analysis results (e.g., prolonged sitting risk score, activity variation score, sleep anomaly score) from these inputs. For example, for desk work, prolonged sitting risk is analyzed in detail; for physical labor, activity variation and physical load are analyzed in detail; for night shifts, sleep patterns and stress levels are analyzed in detail. The AI model is trained using loss functions such as occupation adaptation loss and analysis accuracy loss to maximize living environment adaptation accuracy and analysis accuracy. Training data includes health datasets labeled with occupation and living environment, and data augmentation such as shifting of work time and addition of noise to activity amount is applied. The output of the AI model is passed to the analysis pipeline control module, and after rule-based branching (e.g., sitting time over 8 hours results in detailed analysis) and threshold judgment, it is reflected in the actual analysis flow. As a technical effect, the present analysis unit, unlike conventional uniform analysis processing, can analyze the user's living environment and occupational characteristics with high accuracy and dynamically select the optimal analysis algorithm and level of detail, thereby simultaneously improving risk assessment accuracy, computational efficiency, and user satisfaction. This is useful for occupation-based health risk management, home health monitoring, corporate health management support, and risk assessment for insurance companies.

The proposal unit is capable of considering the user's family history and genetic information when proposing meals or actions based on the user's health condition. For example, when the user has a family history of diabetes, the proposal unit proposes meals effective for diabetes prevention. Additionally, the proposal unit can propose actions to reduce the risk of specific diseases based on the user's genetic information. Furthermore, the proposal unit can propose regular health checks based on the user's family history. By considering the user's family history and genetic information, proposals can be made that support more effective health management. Specifically, the present proposal unit integrates the user's family history data (e.g., family disease history vector [10], onset age list [5]), genetic information data (e.g., disease risk gene polymorphism vector [50]), health condition data (e.g., blood glucose level [7,1440], GFR [7], blood pressure [14]), and user attributes (age, gender, lifestyle), and inputs them to a genetic and family history consideration proposal AI model. The AI model uses disease risk estimation algorithms (e.g., multivariate logistic regression, deep neural networks) and rule-based recommendation engines (e.g., regular checkup recommendations based on family history, meal and exercise proposals based on genotype) in combination. Input examples to the AI model include (1) family disease history vector [10], (2) gene polymorphism vector [50], (3) health indicator vector [10], and (4) user attribute vector [5]. The AI model outputs (a) disease risk score (0.0-1.0), (b) recommended proposal content (output examples: diabetes prevention meals, strengthening exercise habits, regular health checks), and (c) recommended reason text (e.g., based on family history and genetic information) from these inputs. For example, if the user has a family history of diabetes and a genotype with high diabetes risk, the AI model recommends diabetes prevention-focused meal and exercise proposals and regular blood glucose measurement. The AI model is trained using loss functions such as disease risk estimation loss and proposal accuracy loss to maximize risk estimation accuracy and proposal optimization accuracy. Training data includes health management datasets with family history, genetic information, and health indicators, and data augmentation such as shuffling of family history and addition of noise to gene polymorphisms is applied. The output of the AI model is passed to the proposal generation module, and after rule-based branching (e.g., disease risk of 0.7 or higher results in regular checkup recommendation) and threshold judgment, it is reflected in the actual proposal content. As a technical effect, the present proposal unit, unlike conventional uniform proposal generation, can analyze each user's family history, genetic information, and health condition with high accuracy and realize personalized proposals directly linked to disease risk reduction, thereby simultaneously achieving health maintenance, disease prevention, medical cost reduction, and improvement of user satisfaction. This can be widely used in hereditary disease prevention programs, home health management, corporate health management support, and risk reduction measures for insurance companies.

The provision unit can select the optimal provision method by taking into account the user's device information when providing the proposed content to the user. For example, if the user is using a smartphone, the provision unit selects a provision method tailored to the screen size. If the user is using a tablet, the provision unit can select a provision method optimized for a larger screen. Furthermore, if the user is using a smartwatch, the provision unit can select a concise and highly visible provision method. By considering the user's device information, the optimal provision method can be selected. Specifically, the provision unit acquires the user's device information (e.g., device type ID, screen size float, OS version, notification permission setting, communication environment label) in real time and inputs it into a device optimization AI model. The device optimization AI model uses fully connected neural networks or decision tree models and receives as input (1) device type ID (e.g., smartphone ID1, tablet ID2, smartwatch ID3), (2) screen size float, (3) OS version, (4) notification permission setting binary, and (5) communication environment label (e.g., Wi-Fi, 4G, 5G). The AI model outputs (a) recommended provision method ID (output examples: push notification ID1, email ID2, in-app message ID3), (b) recommended UI layout (output examples: simple, detailed, graph-centric), and (c) recommended data volume (output examples: text only, with images, with videos). For example, if the user is using a smartwatch, the provision unit immediately sends a simple text notification only. In the case of a tablet, it sends an email containing detailed graphs and images. The AI model is trained to maximize the accuracy of optimal provision method selection using loss functions such as provision method selection loss and UI optimization loss. The training data includes user response datasets under various device environments, and data augmentation such as device type shuffling and communication environment noise addition is applied. The output of the AI model is passed to the provision scheduler, and through rule-based branching (e.g., simple UI for screen sizes less than 3 inches) and threshold determination, it is reflected in the actual generation of notifications, emails, and in-app messages. As a technical effect, the provision unit, unlike conventional uniform information provision, analyzes the user's device characteristics with high precision and dynamically selects the optimal provision method, UI, and data volume, thereby improving user information receptivity, transmission efficiency, and satisfaction. As a result, it is useful for home health management apps, remote medical support systems, corporate health management platforms, and insurance company risk notification systems.

The following is a brief description of the processing flow of Example of the Embodiment. Specifically, the system utilizes AI models at each stage of health data collection, analysis, proposal, and provision, dynamically optimizing the processing content according to the user's individual characteristics and circumstances. First, the collection unit acquires various data in real time, including the user's health data (e.g., blood glucose level [7,1440], GFR [7], blood pressure [14], heart rate [86400], body temperature [24]), lifestyle data (e.g., jogging time, meal time, sleep time), emotion data (e.g., facial expression images, voice waveforms, heart rate variability, skin electrical activity), geographic location information (e.g., GPS coordinates, altitude, facility ID), and device information (e.g., device type, screen size), and inputs them into various AI models (e.g., emotion estimation AI, lifestyle analysis AI, geographic information analysis AI, device optimization AI). Each AI model generates outputs such as (a) emotion labels and intensity scores, (b) behavior pattern labels, (c) current location categories, and (d) recommended collection timing, frequency, data type, and UI layout, and passes them to the collection scheduler or sensor control module. Next, the analysis unit integrates the collected multidimensional health data with user attributes, living environment, occupation, family history, and genetic information, and inputs them into anomaly detection AI models and living environment adaptive analysis AI models. The analysis unit outputs (a) anomaly scores and anomaly detection labels, (b) risk evaluation scores, and (c) recommended analysis algorithms, detail levels, and order, and passes them to the analysis pipeline control module. The proposal unit integrates analysis results, user preferences, allergies, family history, genetic information, feedback, and emotion data, and inputs them into a personalized proposal AI model. The proposal unit outputs (a) recommended proposal content and reason text, (b) recommended improvement actions, and (c) recommended proposal detail level and order, and passes them to the proposal generation module. The provision unit integrates proposal content, user emotion, device information, and behavioral history, and inputs them into a provision method optimization AI model. The provision unit outputs (a) recommended provision method ID, UI layout, data volume, and timing, (b) recommended notification means (e.g., push notification, email, in-app message), and (c) recommended provision priority, and passes them to the provision scheduler. Each AI model is trained on GPU clusters using loss functions such as classification loss, regression loss, and optimization loss, aiming to maximize accuracy, efficiency, and user experience. Training data includes health datasets labeled by medical professionals, and datasets with lifestyle, emotion, device, and geographic information, with data augmentation such as time series shifting, noise addition, and paraphrase generation applied. As a technical effect, the system, unlike conventional uniform health management systems, dynamically optimizes the entire processing flow according to each user's characteristics, circumstances, and environment, thereby simultaneously achieving prevention of missed health indicators, early detection of anomalies, minimization of user burden, improvement of satisfaction, and operational efficiency of the system. As a result, it can be applied to various fields such as home health monitoring, remote medical support, corporate health management platforms, insurance company risk evaluation systems, athlete performance management, and care support systems.

Step 1: The collection unit collects the user's health data. The user's health data includes blood glucose level, GFR, blood pressure, heart rate, body temperature, and so on. The collection unit can collect data using sensors, and can also collect data manually entered by the user. For example, data can be collected using sensors that measure blood glucose level or sensors that measure GFR. Step 2: The analysis unit analyzes the data collected by the collection unit. The analysis is performed using statistical analysis or machine learning algorithms. For example, statistical analysis can be used to analyze trends in the user's health data, or machine learning algorithms can be used to analyze correlations in the data. Step 3: The proposal unit makes optimal proposals based on the analysis results obtained by the analysis unit. Proposals include meal proposals, exercise proposals, stress management proposals, and so on. For example, based on the user's health condition, proposals for low-carbohydrate meals or exercise can be made. Step 4: The provision unit provides the content proposed by the proposal unit to the user. Provision is performed by methods such as notifications, emails, or in-app messages. For example, the proposed content can be provided to the user as a notification, or provided as an email or in-app message. Specifically, in Step 1, the system automatically acquires multidimensional health data such as blood glucose level [7,1440], GFR [7], blood pressure [14], heart rate [86400], and body temperature [24] from sensors, and simultaneously acquires lifestyle data (e.g., jogging time, meal time), emotion data (e.g., facial expression images, voice waveforms), geographic location information (e.g., GPS coordinates, altitude), and device information (e.g., device type, screen size). The collection unit inputs these data into AI models such as emotion estimation AI, lifestyle analysis AI, geographic information analysis AI, and device optimization AI, and outputs recommended collection timing, frequency, data type, and UI layout, which are reflected in sensor control and data collection planning. In Step 2, the analysis unit integrates the collected data with user attributes, living environment, occupation, family history, and genetic information, inputs them into anomaly detection AI and living environment adaptive analysis AI, and outputs anomaly scores, risk evaluation, recommended analysis algorithms, detail level, and order, which are reflected in analysis pipeline control. In Step 3, the proposal unit inputs analysis results, preferences, allergies, family history, genetic information, feedback, and emotion data into personalized proposal AI, and outputs recommended proposal content, reasons, improvement actions, detail level, and order, which are passed to the proposal generation module. In Step 4, the provision unit inputs proposal content, emotion, device information, and behavioral history into provision method optimization AI, and outputs recommended provision method, UI layout, data volume, timing, notification means, and priority, which are reflected in the provision scheduler. Each AI model is trained on GPU clusters using classification loss, regression loss, and optimization loss as loss functions, aiming to maximize accuracy, efficiency, and user experience. Training data includes health datasets labeled by medical professionals, and datasets with lifestyle, emotion, device, and geographic information, with data augmentation such as time series shifting, noise addition, and paraphrase generation applied. As a technical effect, the system, unlike conventional uniform health management systems, dynamically optimizes the entire processing flow according to each user's characteristics, circumstances, and environment, thereby simultaneously achieving prevention of missed health indicators, early detection of anomalies, minimization of user burden, improvement of satisfaction, and operational efficiency of the system. As a result, it can be applied to various fields such as home health monitoring, remote medical support, corporate health management platforms, insurance company risk evaluation systems, athlete performance management, and care support systems.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned collection unit, analysis unit, proposal unit, and provision unit is implemented, for example, by at least one of a smart device 14 and a data processing apparatus 12. For example, the collection unit collects health data through sensors of the smart device 14 or manual input by the user. The analysis unit is implemented, for example, by a specific processing unit 290 of the data processing apparatus 12, and analyzes the collected data using statistical analysis or machine learning algorithms. The proposal unit is implemented, for example, by the specific processing unit 290 of the data processing apparatus 12, and makes proposals for optimal meals or exercise based on the analysis results. The provision unit is implemented, for example, by a control unit 46A of the smart device 14, and provides the proposed content to the user as notifications, emails, or in-app messages. The correspondence between each unit and the apparatus or control unit is not limited to the examples described above, and various modifications are possible.

### Second Embodiment

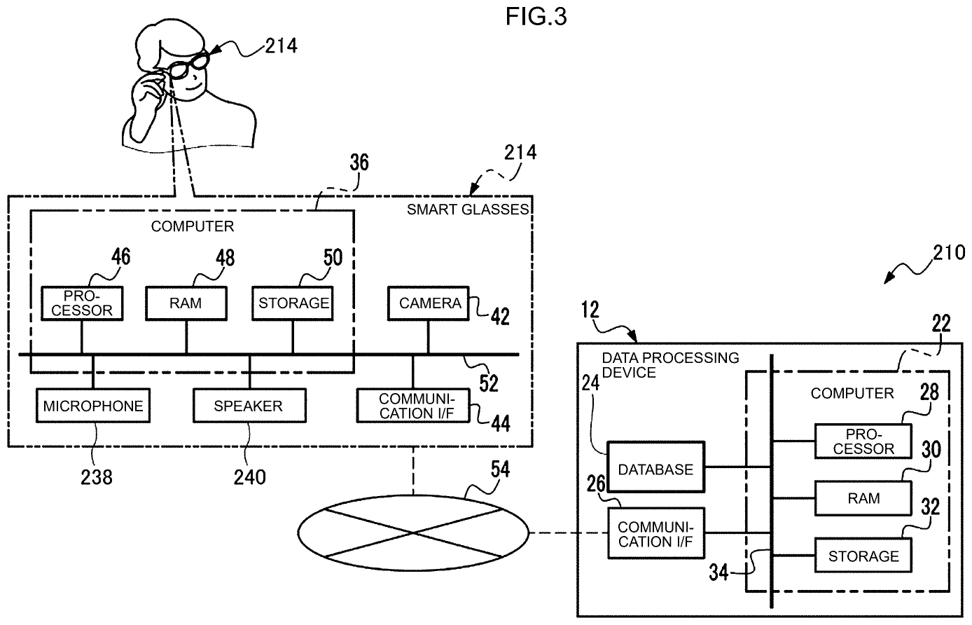

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

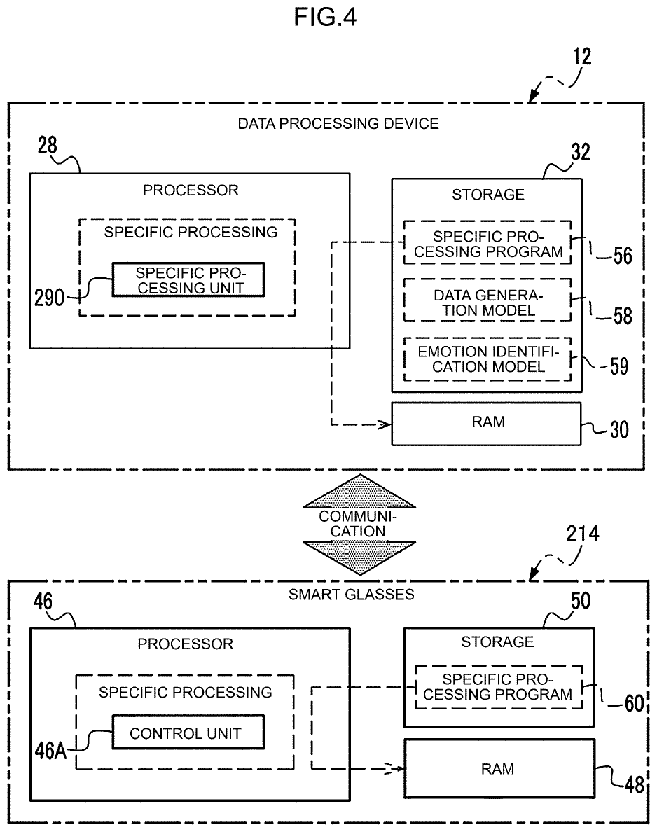

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned collection unit, analysis unit, proposal unit, and provision unit is implemented, for example, by at least one of smart glasses 214 and a data processing apparatus 12. For example, the collection unit collects health data through sensors of the smart glasses 214 or manual input by the user. The analysis unit is implemented, for example, by a specific processing unit 290 of the data processing apparatus 12, and analyzes the collected data using statistical analysis or machine learning algorithms. The proposal unit is implemented, for example, by the specific processing unit 290 of the data processing apparatus 12, and makes proposals for optimal meals or exercise based on the analysis results. The provision unit is implemented, for example, by a control unit 46A of the smart glasses 214, and provides the proposed content to the user as notifications, emails, or in-app messages. The correspondence between each unit and the apparatus or control unit is not limited to the examples described above, and various modifications are possible.

### Third Embodiment

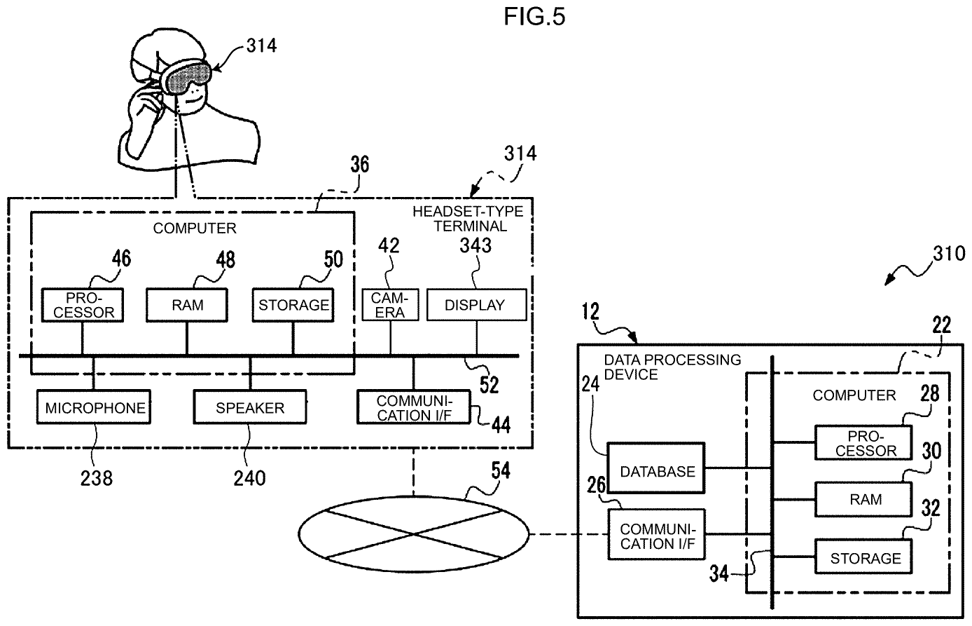

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

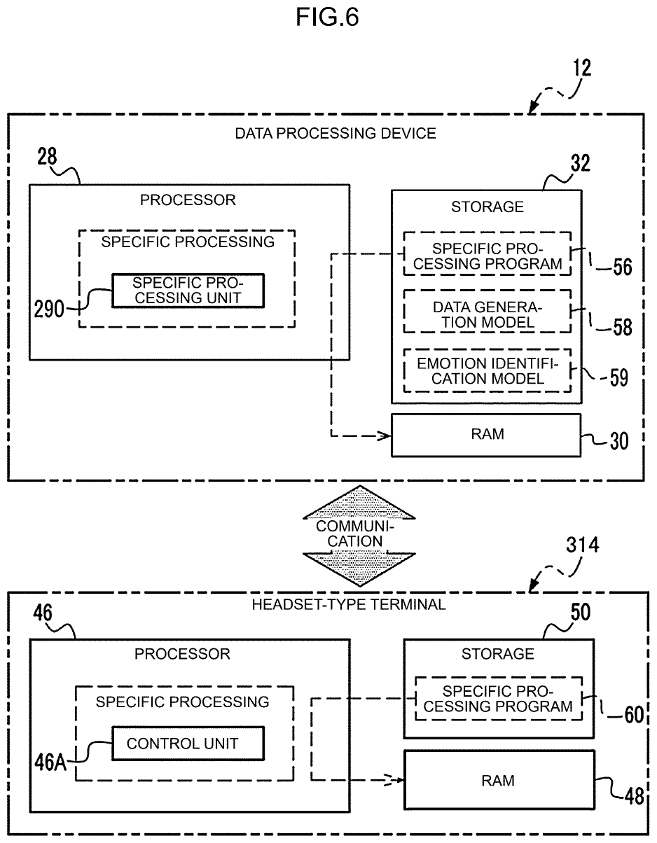

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned collection unit, analysis unit, proposal unit, and provision unit is implemented, for example, by at least one of a headset-type terminal 314 and a data processing apparatus 12. For example, the collection unit collects health data through sensors of the headset-type terminal 314 or manual input by the user. The analysis unit is implemented, for example, by a specific processing unit 290 of the data processing apparatus 12, and analyzes the collected data using statistical analysis or machine learning algorithms. The proposal unit is implemented, for example, by the specific processing unit 290 of the data processing apparatus 12, and makes proposals for optimal meals or exercise based on the analysis results. The provision unit is implemented, for example, by a control unit 46A of the headset-type terminal 314, and provides the proposed content to the user as notifications, emails, or in-app messages. The correspondence between each unit and the apparatus or control unit is not limited to the examples described above, and various modifications are possible.

### Fourth Embodiment

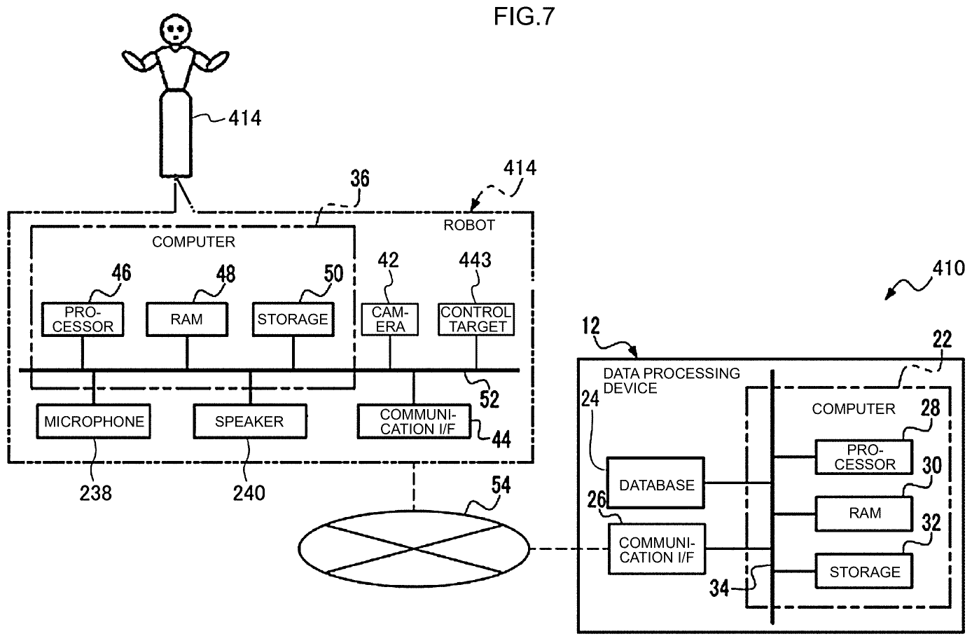

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

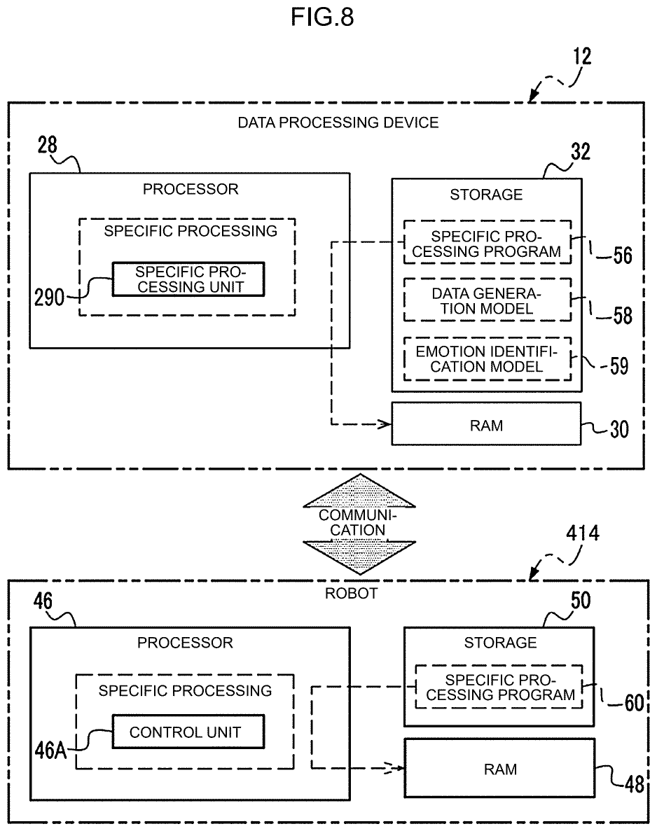

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned collection unit, analysis unit, proposal unit, and provision unit is implemented, for example, by at least one of a robot 414 and a data processing apparatus 12. For example, the collection unit collects health data through sensors of the robot 414 or manual input by the user. The analysis unit is implemented, for example, by a specific processing unit 290 of the data processing apparatus 12, and analyzes the collected data using statistical analysis or machine learning algorithms. The proposal unit is implemented, for example, by the specific processing unit 290 of the data processing apparatus 12, and makes proposals for optimal meals or exercise based on the analysis results. The provision unit is implemented, for example, by a control unit 46A of the robot 414, and provides the proposed content to the user as notifications, emails, or in-app messages. The correspondence between each unit and the apparatus or control unit is not limited to the examples described above, and various modifications are possible.

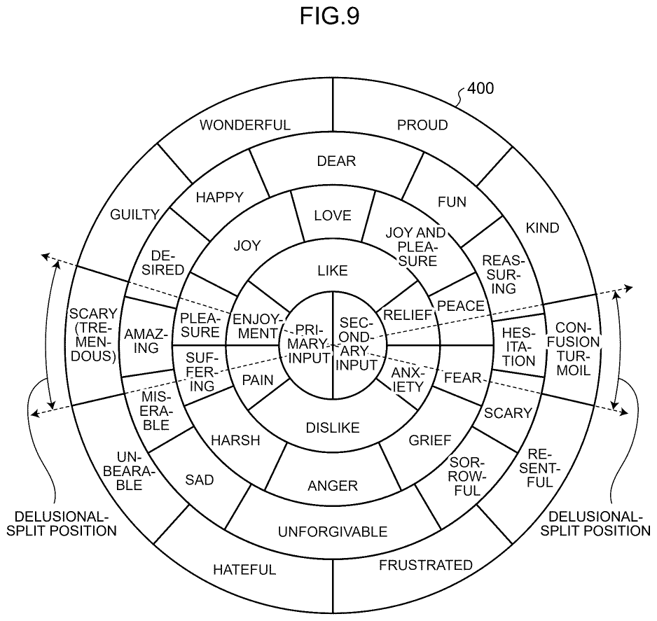

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

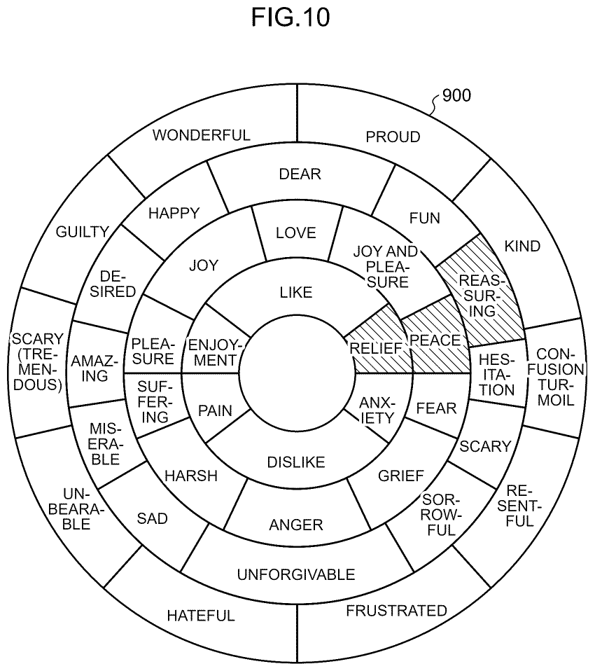

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### (Supplementary Note 1)

A system comprising: a collection unit configured to collect health data of a user; an analysis unit configured to analyze data collected by the collection unit; a proposal unit configured to make proposals based on analysis results obtained by the analysis unit; and a provision unit configured to provide the content proposed by the proposal unit to the user.

### (Supplementary Note 2)

The system according to Supplementary Note 1, wherein the collection unit is configured to collect health data of blood glucose level or GFR.

### (Supplementary Note 3)

The system according to Supplementary Note 1, wherein the analysis unit is configured to analyze the collected data and identify a relationship between the user's behavior and numerical fluctuations.

### (Supplementary Note 4)

The system according to Supplementary Note 1, wherein the proposal unit is configured to propose meals or actions based on the user's health condition.

### (Supplementary Note 5)

The system according to Supplementary Note 1, wherein the provision unit is configured to provide the proposed content to the user.

### (Supplementary Note 6)

The system according to Supplementary Note 1, wherein the proposal unit is configured to receive user feedback and improve the proposed content.

### (Supplementary Note 7)

The system according to Supplementary Note 1, wherein the collection unit is configured to estimate the user's emotion and adjust the timing of health data collection based on the estimated emotion of the user.

### (Supplementary Note 8)

The system according to Supplementary Note 1, wherein the collection unit is configured to analyze the user's past health data and select a collection method.

### (Supplementary Note 9)

The system according to Supplementary Note 1, wherein the collection unit is configured to perform filtering based on the user's current living situation or areas of interest at the time of health data collection.

### (Supplementary Note 10)

The system according to Supplementary Note 1, wherein the collection unit is configured to estimate the user's emotion and determine the priority of health data to be collected based on the estimated emotion of the user.

### (Supplementary Note 11)

The system according to Supplementary Note 1, wherein the collection unit is configured to preferentially collect highly relevant data based on the user's geographic location information at the time of health data collection.

### (Supplementary Note 12)

The system according to Supplementary Note 1, wherein the collection unit is configured to analyze the user's social media activity and collect relevant data at the time of health data collection.

### (Supplementary Note 13)

The system according to Supplementary Note 1, wherein the analysis unit is configured to estimate the user's emotion and adjust the method of presenting analysis based on the estimated emotion of the user.

### (Supplementary Note 14)

The system according to Supplementary Note 1, wherein the analysis unit is configured to adjust the level of detail of analysis based on the importance of the health data during analysis.

### (Supplementary Note 15)

The system according to Supplementary Note 1, wherein the analysis unit is configured to apply different analysis algorithms according to the category of health data during analysis.

### (Supplementary Note 16)

The system according to Supplementary Note 1, wherein the analysis unit is configured to estimate the user's emotion and adjust the length of analysis based on the estimated emotion of the user.

### (Supplementary Note 17)

The system according to Supplementary Note 1, wherein the analysis unit is configured to determine the priority of analysis based on the timing of health data collection during analysis.

### (Supplementary Note 18)

The system according to Supplementary Note 1, wherein the analysis unit is configured to adjust the order of analysis based on the relevance of health data during analysis.

### (Supplementary Note 19)

The system according to Supplementary Note 1, wherein the proposal unit is configured to estimate the user's emotion and adjust the method of presenting proposals based on the estimated emotion of the user.

### (Supplementary Note 20)

The system according to Supplementary Note 1, wherein the proposal unit is configured to adjust the level of detail of proposals based on the importance of the health condition at the time of proposal.

### (Supplementary Note 21)

The system according to Supplementary Note 1, wherein the proposal unit is configured to apply different proposal algorithms according to the category of health condition at the time of proposal.

### (Supplementary Note 22)

The system according to Supplementary Note 1, wherein the proposal unit is configured to estimate the user's emotion and adjust the length of proposals based on the estimated emotion of the user.

### (Supplementary Note 23)

The system according to Supplementary Note 1, wherein the proposal unit is configured to determine the priority of proposals based on the timing of health data collection at the time of proposal.

### (Supplementary Note 24)

The system according to Supplementary Note 1, wherein the proposal unit is configured to adjust the order of proposals based on the relevance of health data at the time of proposal.

### (Supplementary Note 25)

The system according to Supplementary Note 1, wherein the provision unit is configured to estimate the user's emotion and adjust the method of provision based on the estimated emotion of the user.

### (Supplementary Note 26)

The system according to Supplementary Note 1, wherein the provision unit is configured to select a method of provision based on the user's past behavioral history at the time of provision.

### (Supplementary Note 27)

The system according to Supplementary Note 1, wherein the provision unit is configured to estimate the user's emotion and determine the priority of provision based on the estimated emotion of the user.

### (Supplementary Note 28)

The system according to Supplementary Note 1, wherein the provision unit is configured to select an optimal method of provision in consideration of the user's device information at the time of provision.

What is claimed is:

1. A system comprising:
a communication interface configured to communicate with a client terminal via a packet-switched network;
a memory storing a data generation model obtained by deep learning on a neural network, and an emotion identification model; and
circuitry configured to:
receive, from the client terminal via the communication interface, structured data comprising at least one of time-series numerical data, categorical data, or text data;
estimate an emotion of a user by applying the emotion identification model to sensor data received from the client terminal;
analyze the structured data by inputting the structured data together with the estimated emotion into the data generation model to generate inference data comprising at least one of a classification label, a probability score, or a recommendation text;
generate rendering data for a visualization of the inference data; and
transmit the inference data and the rendering data to the client terminal via the communication interface and the packet-switched network.

2. The system according to claim 1, wherein the structured data comprises health information of a user, the health information comprising at least one of blood glucose level, glomerular filtration rate, blood pressure, heart rate, or body temperature as numerical vectors.

3. The system according to claim 1, wherein the structured data further comprises behavioral data comprising at least one of meal content as one-hot encoded vectors, exercise intensity vectors, or sleep quality scores.

4. The system according to claim 1, wherein the circuitry is further configured to preprocess the structured data by performing at least one of missing value imputation, outlier removal, or normalization before inputting the structured data into the data generation model.

5. The system according to claim 1, wherein the data generation model comprises a multimodal neural network configured to integratively analyze the text data and the time-series numerical data.

6. The system according to claim 1, wherein the inference data comprises a prediction vector for a health indicator over a future time period, and wherein the circuitry is further configured to perform threshold determination based on the prediction vector to generate an alert when the prediction vector exceeds a predetermined threshold.

7. The system according to claim 1, wherein the circuitry is further configured to adjust a timing of receiving the structured data from the client terminal based on the estimated emotion, such that when the estimated emotion indicates stress, the circuitry delays the receiving, and when the estimated emotion indicates relaxation, the circuitry receives the structured data immediately.

8. The system according to claim 1, wherein the circuitry is further configured to determine a priority of the structured data to be received based on the estimated emotion, such that when the estimated emotion indicates stress, the circuitry prioritizes receiving structured data having a high importance attribute.

9. The system according to claim 1, wherein the circuitry is further configured to adjust a level of detail of the inference data based on the estimated emotion, such that when the estimated emotion indicates stress, the circuitry generates concise inference data, and when the estimated emotion indicates relaxation, the circuitry generates detailed inference data.

10. The system according to claim 1, wherein the circuitry is further configured to adjust an expression style of the inference data based on the estimated emotion, such that when the estimated emotion indicates stress, the inference data is generated in a simplified expression style, and when the estimated emotion indicates relaxation, the inference data is generated in a detailed expression style.

11. The system according to claim 1, wherein the circuitry is further configured to calculate an importance score for each item of the structured data and to adjust a level of detail of analysis based on the importance score, such that detailed analysis is performed for structured data having a high importance score and simplified analysis is performed for structured data having a low importance score.

12. The system according to claim 1, wherein the circuitry is further configured to apply different analysis algorithms according to a category of the structured data.

13. The system according to claim 1, wherein the circuitry is further configured to receive geographic location information of the user from the client terminal and to preferentially receive structured data associated with a geographic region corresponding to the geographic location information.

14. The system according to claim 1, wherein the circuitry is further configured to receive social media activity data of the user from the client terminal, analyze the social media activity data using a natural language processing model to extract status information, and adjust a type of the structured data to be received based on the extracted status information.

15. The system according to claim 1, wherein the circuitry is further configured to select a method of transmitting the inference data to the client terminal based on at least one of the estimated emotion or a usage history of the user, the method comprising at least one of a push notification, an email, or an in-app message.

16. The system according to claim 1, wherein the circuitry is further configured to select an optimal transmission method for the inference data based on device information of the client terminal received via the communication interface, the device information comprising at least one of a device type, a screen size, or an operating system version.

17. The system according to claim 1, wherein the rendering data comprises parameters for at least one of a time-series graph, a radar chart, or a heat map for visualizing the inference data.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model; and
circuitry configured to:
receive, from the client terminal via the communication interface, structured data comprising at least one of time-series numerical data representing health indicators, categorical data representing lifestyle habits, or text data representing user queries;
preprocess the structured data by performing at least one of missing value imputation, outlier removal, or normalization;
estimate an emotion of a user by applying the emotion identification model to at least one of voice data captured by the microphone or image data captured by the camera;
analyze the preprocessed structured data by inputting the preprocessed structured data together with the estimated emotion into the data generation model to generate inference data comprising at least one of a classification label, a probability score, or a recommendation text;
adjust at least one of a level of detail, an expression style, or a length of the inference data based on the estimated emotion;
generate rendering data for a visualization of the inference data, the rendering data comprising parameters for at least one of a time-series graph, a radar chart, or a heat map; and
transmit the inference data and the rendering data to the client terminal via the communication interface, the inference data and the rendering data causing the client terminal to present the inference data and the visualization to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network and an emotion identification model, the method comprising:
receiving, from a client terminal via the communication interface and a packet-switched network, structured data comprising at least one of time-series numerical data, categorical data, or text data;
estimating an emotion of a user by applying the emotion identification model to sensor data received from the client terminal;
analyzing the structured data by inputting the structured data together with the estimated emotion into the data generation model to generate inference data comprising at least one of a classification label, a probability score, or a recommendation text;
generating rendering data for a visualization of the inference data; and
transmitting the inference data and the rendering data to the client terminal via the communication interface and the packet-switched network.
